(19) United States
(12) Patent Application Publication
(10) Pub. No.: US 2026/0216142 A1
(43) Pub. Date: Jul. 30, 2026

(54) USES OF ZLN-005 AND RELATED COMPOUNDS

(71) Applicant: Satoshi GOJO, Kyoto (JP)

(72) Inventor: Satoshi GOJO, Kyoto (JP)

(21) Appl. No.: 19/130,852

(22) PCT Filed: Nov. 17, 2023
(86) PCT No.: PCT/IB2023/061651

§ 371 Date: May 16, 2025

Related U.S. Application Data
(60) Provisional application No. 63/426,426, filed on Nov. 18, 2022.
(60) Provisional application No. 63/482,384, filed on Jan. 31, 2023.

(51) Int. Cl. A61K 31/4184; A61P 31/00; A61P 31/04; A61P 37/02

(52) U.S. Cl. CPC A61K 31/4184; A61P 31/00; A61P 31/04; A61P 37/02

(57) ABSTRACT

Methods of treating diseases and disorders associated with impaired lysosomal acidification, methods for increasing lysosomal acidity, and methods of treating sepsis, infection, and diseases and disorders associated with V-ATPase dysfunction with ZLN-005 and other compounds of Formula (I), and salts, hydrates, deuterated analogs, and fluorinated analogs thereof.

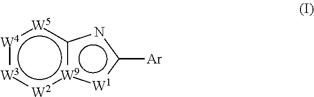

### 1. CROSS-REFERENCE TO RELATED APPLICATIONS

This application claims the priority benefit of U.S. provisional application No. 63/426,426, filed Nov. 18, 2022, and U.S. provisional application No. 63/482,384, filed Jan. 31, 2023, the contents of which are incorporated herein in their entireties by reference thereto.

### 2. BACKGROUND

Impaired lysosomal acidification can lead to impaired phagocyte function. Impaired lysosomal acidification and impaired phagocyte function have been implicated in numerous diseases and disorders, including some neurodegenerative disorders, diseases and disorders associated with V-ATPase dysfunction, sepsis and infection (Colacurio and Nixon, 2016, Ageing Rev. 2016 32:75-88; Chiswick et al., 2015, J. Immunol. 195(8):3793-802; Danikas et al., 2008, Clinical and Experimental Immunology 154:87-97).

New treatments for treating diseases and disorders associated with impaired lysosomal acidification are needed.

### 3. SUMMARY

The present disclosure is based, in part, on the discovery that 2-(4-tert-butylphenyl)-1H-benzimidazole (ZLN-005; also known as TQS-168), an activator of Ppargc1α (PGC-1α) expression, increases phagocytic activity of phagocytes in an in vitro assay, promotes lysosomal acidification in an in vitro assay, and reduces mortality in an in vivo sepsis model in which mortality is known to be associated with impaired lysosomal acidification. ZLN-005 has the following structure:

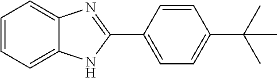 [SMILES: CC(C)(C)c1ccc(-c2nc3ccccc3[nH]2)cc1]

The assays indicate that ZLN-005 and related compounds can be used to increase lysosomal acidification, e.g., in a subject's phagocytic cells such as monocytes, and (without being bound by theory) be used to treat diseases and disorders associated with impaired lysosomal acidification such as sepsis accompanied by suppressed innate immunity, infections, and diseases and disorders associated with V-ATPase dysfunction.

Accordingly, in one aspect, the disclosure provides methods of treating a subject having a disease or disorder associated with impaired lysosomal acidification comprising administering to the subject a therapeutically effective amount of an agent which is ZLN-005 or other compound of Formula (I):

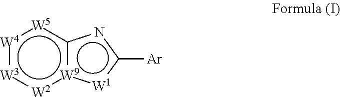

or a salt, hydrate, deuterated analog, or fluorinated analog thereof, wherein:
Ar is

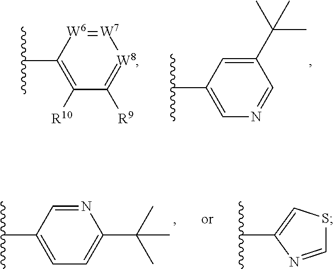

W¹ is N—R¹, O, or S, or when W⁹ is N, W¹ may additionally be C—R⁵⁰;
W² is C—R² or N;
W³ is C—R³ or N;
W⁴ is C—R⁴ or N;
W⁵ is C—R⁵ or N;
W⁶ is C—R⁶ or N;
W⁷ is C—R⁷ or N;
W⁸ is C—R⁸ or N;
W⁹ is C, or when W¹ is C—R⁵⁰, W⁹ may be N;
R¹ is H, (C₁-C₃)alkyl, CH₂OC(═O)R³⁰, CH₂OP(═O)OR⁴OR⁴¹, C(═O)OR⁴², or C(═O)R⁴³;
each of R², R³, R⁴, and R⁵ is independently hydrogen, deuterium, halogen, perfluoro(C₁-C₄)alkyl, (C₁-C₄)alkyl, (C₁-C₄)alkoxy, perfluoro(C₁-C₄)alkoxy, (C₁-C₄)acyl, (C₁₋₄)alkoxy(C₁-C₄)alkyl, hydroxy(C₁-C₄)alkyl, hydroxy, carboxy, (C₁-C₄)alkoxycarbonylamino, carboxamido, (C₁-C₄)alkylaminocarbonyl, cyano, acetoxy, nitro, amino, (C₁-C₄)alkylamino, di(C₁-C₄)alkylamino, mercapto, (C₁-C₄)alkylthio, aminosulfonyl, (C₁-C₄)alkylsulfonyl, or (C₁-C₄)acylamino;
each of R⁶ and R¹⁰ is independently hydrogen, deuterium, halo, (C₁-C₃)alkyl, perfluoro(C₁-C₃)alkyl, hydroxy, (C₁-C₃)alkoxy, perfluoro(C₁-C₃)alkoxy, or amino;
each of R⁷ and R⁸ is independently hydrogen, deuterium, hydroxy, cyano, amino, halogen, halo(C₁-C₄)alkyl, (C₁-C₄)alkoxy, halo(C₁-C₄)alkoxy,

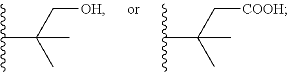

R⁸ is hydrogen, deuterium, halogen, halo(C₁-C₄)alkyl, (C₁-C₄)alkyl, (C₁-C₄)alkoxy, halo(C₁-C₄)alkoxy, cyano, phenyl, phenoxy, benzyloxy, amino,

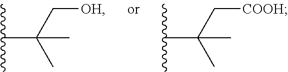

R³⁰ is (C₁-C₁₀)hydrocarbyl, (C₁-C₁₀)hydrocarbyl substituted with amino, (C₁-C₁₀)hydrocarbyl substituted with (C₁-C₄)hydrocarbyl, (C₁-C₁₀)hydrocarbyl substituted with carboxyl, carboxy, (C₁-C₆)alkoxycarbonyl, (C₁-C₆)alkoxycarbonylamino, methylthio, heterocycyl, (C₁-C₁₀)oxaalkyl, CHR⁴⁴NHR⁴⁵ and guanidine;
each of R⁴⁰ and R⁴¹ is independently hydrogen or (C₁-C₆)hydrocarbyl;
R⁴² is (C₁-C₅)alkyl;
R⁴³ is (C₁-C₃)alkyl,
R⁴⁴ is a naturally occurring amino acid sidechain;
R⁴⁵ is H, methyl, or (C₁-C₄)alkoxycarbonyl; and
R⁵⁰ is H or (C₁-C₃)alkyl.

In another aspect, the disclosure provides methods for increasing lysosomal acidity in a subject comprising administering to the subject an amount of an agent effective to increase lysosomal acidity in the subject, wherein the agent is a compound of Formula (I) (e.g., ZLN-005) or a salt, hydrate, deuterated analog, or fluorinated analog thereof.

In another aspect, the disclosure provides methods of treating a subject having sepsis, an infection, or a disease or disorder associated with V-ATPase dysfunction, comprising administering to the subject a therapeutically effective amount of an agent, wherein the agent is a compound of Formula (I) (e.g., ZLN-005) or a salt, hydrate, deuterated analog, or fluorinated analog thereof. In some embodiments, the sepsis is polymicrobial sepsis. In other embodiments, the sepsis is unimicrobial sepsis.

Further features compounds of Formula (I) and salts, hydrates, deuterated analogs, and fluorinated analogs thereof are described in Section 5.2 and numbered embodiments 1 to 3 and 90 to 103, infra.

Compounds of Formula (I) and salts, hydrates, deuterated analogs, and fluorinated analogs thereof can be administered in a pharmaceutical composition comprising the compound of Formula (I) or a salt, hydrate, deuterated analog, or fluorinated analog thereof.

Exemplary features of pharmaceutical compositions are described in Section 5.3, infra.

Further features of the methods of the disclosure are described in Section 5.4 and numbered embodiments 1 to 106, infra.

### 4. BRIEF DESCRIPTION OF THE FIGURES

FIGS. 1A-1D show a study timeline for a cecal ligation and puncture (CLP) model of sepsis using C57BL/6 mice treated intraperitoneally with vehicle (DMSO) or ZLN-005 every 24 hours CLP (FIG. 1A); Kaplan-Meier survival proportions for mice in the CLP model of sepsis treated with ZLN-005 or vehicle (n=9) (FIG. 1B); mRNA expression of Pgc1α & Tfam at 24 hours post-CLP (n=6) (FIG. 1C); and mRNA expression of proinflammatory cytokines at 24 hours post-CLP (n=6) (FIG. 1D) (Example 1 and Example 5). P values ** <0.0001, P values *0.0001 to 0.05.

FIG. 2 shows phagocytic activity of macrophages treated with ZLN-005 (Example 2).

FIG. 3 shows light microscopy images (top row) and fluorescence microscopy images using a pH sensitive dye (bottom row) of macrophages treated with ZLN-005 or vehicle (DMSO) (Example 3).

FIG. 4 shows lysosomal acidity of macrophages treated with ZLN-005 or vehicle (DMSO) (Example 3).

FIG. 5 shows light microscopy images (top row) and fluorescence microscopy images (bottom row) of peritoneal cells collected from mice in the CLP model of sepsis treated with ZLN-005 or vehicle (DMSO) following incubation with fluorescent beads (Example 4).

FIG. 6 shows relative phagocytic activity of peritoneal cells collected from mice in the CLP model of sepsis treated with ZLN-005 or vehicle (DMSO) (Example 4).

FIG. 7 shows light microscopy images (top row) and fluorescence microscopy images using a pH sensitive dye (bottom row) of peritoneal cells from mice in the CLP model of sepsis treated with ZLN-005 or vehicle (DMSO) (Example 4).

FIG. 8 shows lysosomal acidity of peritoneal cells from mice in the CLP model of sepsis treated with ZLN-005 or vehicle (DMSO) (Example 4).

FIG. 9 shows peritoneal bacteria levels from mice in the CLP model of sepsis treated with ZLN-005 or vehicle (DMSO) (Example 4).

FIGS. 10A-10C show organ protective effect of ZLN-005 in the CLP sepsis model (Example 5). FIG. 10A: ejection fraction, left ventricular diameter and left ventricular diastolic wall thickness at 24 hours post-CLP (n=4). FIG. 10B: Histological score of the liver in mice at 24 hours post-CLP (n=3). FIG. 10C: Histological score of the lung in mice at 24 hours post-CLP (n=3). P values *0.0001 to 0.05.

FIGS. 11A-11B show changes in the oxygen consumption rate (OCR) (FIG. 11A) and extracellular acidification rate (ECAR) (FIG. 11B) of THP-1 cells in an inflammation model, as measured using flux analysis (Example 5). O: oligomycin, F: FCCP, A&R: antimycin and rotenone, G: glucose, 2-DG: 2-deoxy-D-glucose (n=5). Each index of glycolysis was calculated from the ECAR measurement results. Each index of mitochondrial respiratory function was calculated from the OCR, glycolysis was calculated from the ECAR measurement results. P values **<0.0001, P values *0.0001 to 0.05.

FIGS. 12A-12G show mitochondrial functional alterations by ZLN-005 (Example 5). FIG. 12A: mRNA expression of PGC1α and TFAM in THP-1 cells in the inflammation model at 24 hours post-LPS administration (n=3). FIG. 12B: absolute copy number of THP-1 in the inflammation model at 24 hours post-LPS administration (n=3). FIG. 12C: MFI ratio of mtROS levels in THP-1 cells in the inflammation model at 24 hours post-LPS administration (n=3). FIG. 12D: MFI ratio of acidic mKeima Red signaling in THP-1 cells in the inflammation model at 0, 4, 8 and 24 hours post-LPS administration (n=3). FIG. 12E: MFI ratio of Δφ of THP-1 cells in the inflammation model at 24 hours post-LPS administration (n=3). FIG. 12F: Mitochondrial morphology of THP-1 cells in the inflammation model at 24 hours post-LPS administration. All bars in the images are 20 μm. The graph shows the mitochondrial footprint, junction pixel and slab pixel areas per cell (n=5). FIG. 12G: Assessment of the mitochondria-lysosome contact site of THP-1 cells in the inflammation model at 0, 1, 2, 8 and 24 hours after LPS administration. All bars in the images are 20 μm. The graph shows the ratio of the contact site area to the total mitochondrial area per cell (n=5). P values *0.0001 to 0.05.

FIGS. 13A-13H show lysosomal functional alterations by ZLN-005 (Example 5). FIG. 13A: mRNA expression of Tfeb and MFI ratio of lysosomal staining of peritoneal cavity cells at 24 hours post-CLP (n=6). FIGS. 13B-13D: assessment of phagocytosis (n=6) (FIG. 13B), lysosomal acidification (FIG. 13C), and cellular ROS (FIG. 13D) in peritoneal cavity cells at 24 hours post-CLP. FIG. 13E: degradation of lysosomally preloaded DQ™ BSA in peritoneal cavity cells at 24 hours post-CLP. The graph shows the MFI ratio of degraded DQ Green BSA and LysoTracker Red. FIG. 13F-13G: mRNA expression of lysosomal hydrolase (FIG. 13F) and autophagy-related genes (FIG. 13G) at 24 hours post-CLP (n=6). FIG. 13H: Western blotting analysis of the autophagy flux in THP-1 cells after 6 hours of exposure to LPS followed by 2 or 6 hours of treatment with the autophagy inhibitor. The graph shows the expression ratio of LC3-II protein corrected by LC3-I protein expression level (n=3). (Con: Control, Baf: Bafilomycin A1, CQ: Chloroquine) P values ** <0.0001, P values * 0.0001 to 0.05.

FIGS. 14A-14E show the effect of ZLN-005 on Tfeb (Example 5). FIG. 14A: Study design to assess the mRNA expression of Tfeb and MFI ratio of lysosomal staining of peritoneal cavity cells at 24 hours post-CLP (n=6). FIGS. 14B-14D: The assessment of phagocytosis (n=6) (FIG. 14B), lysosomal acidification (C), and cellular ROS (D) in peritoneal cavity cells at 24 hours post-CLP. (FIG. 14E) Degradation of lysosomally preloaded DQ™ BSA in peritoneal cavity cells at 24 hours post-CLP. The graph shows the MFI ratio of degraded DQ™ Green BSA and LysoTracker™ Red. FIGS. 14F-14G: mRNA expression of lysosomal hydrolase (FIG. 14F) and autophagy-related genes (FIG. 14G) at 24 hours post-CLP (n=6). FIG. 14H: Western blotting analysis of the autophagy flux in THP-1 cells after 6 hours of exposure to LPS followed by 2 or 6 hours of treatment with the autophagy inhibitor. The graph shows the expression ratio of LC3-II protein corrected by LC3-I protein expression level (n=3). (Con: Control, Baf: Bafilomycin A1, CQ: Chloroquine) P values ** <0.0001, P values * 0.0001 to 0.05.

FIGS. 15A-15B show Western blotting analysis (FIG. 15A) of the molecular pathways of peritoneal cavity cells at 24 hours post-CLP and assessment of lysosomal acidification in THP-1 cells at 5 days post-Wortmannin administration (FIG. 15B) (Example 5). The graph in FIG. 15A shows the expression ratio of each protein corrected by Gapdh protein expression level (n=3). For FIG. 15B, the statistical significance of differences among two groups, with or without ZLN-005 in Wortmannin administration, were evaluated using Kolmogorov-Smimov test. P values **<0.0001, P values * 0.0001 to 0.05.

FIG. 16 shows a graphic schema for a proposed mode of action of ZLN-005 in polymicrobial sepsis. The schema is for illustrative purposes only, and does not limit any aspect or embodiment disclosed herein to a particular mechanism.

FIGS. 17A-17D show the effect of ZLN-005 or vehicle (DMSO) on peritoneal bacteria levels from mice in the CLP model of sepsis (Example 6). FIG. 17A is a diagram that shows the study design. FIG. 17B are photographs that illustrate the changes in bacterial colonies in ascites from sham-treated, CLP and vehicle-treated, and CLP and ZLN-005-treated mice over 2 and 24 hours. FIG. 17C is a bar graph displaying the bacteria count in ascites from sham-treated, CLP and vehicle-treated, and CLP and ZLN-005-treated mice 2 hours after CLP. FIG. 17D is a bar graph displaying the bacteria count in ascites from sham-treated, CLP and vehicle-treated, and CLP and ZLN-005-treated mice 24 hours after CLP.

FIG. 18A-18B show the effect of ZLN-005 on v-ATPase assembly in a CLP mouse model of sepsis (Example 7). FIG. 18A is a diagram that shows the study design. FIG. 18B are graphs that show western blot results as relative protein expression levels for the proteins indicated above each graph.

FIG. 19A-19B show the effect of TRPML1 inhibition on ZLN-005-mediated lysosomal acidification due to LPS stimulation (Example 8). FIG. 19A is a diagram that shows the study design. FIG. 19B shows the pHrod fluorescence levels in different treatment groups.

### 5. DETAILED DESCRIPTION

This disclosure provides novel uses of ZLN-005 and other compounds of Formula (I) (as well as salts, hydrates, deuterated analogs, and fluorinated analogs thereof), for example in methods of treating subjects having impaired lysosomal acidification, methods for increasing lysosomal acidity in a subject, methods of treating subjects having sepsis (e.g., where the subject has suppressed innate immune function, for example associated with impaired lysosomal acidification), methods of treating subjects having an infection, and methods of treating subjects having a disease or disorder associated with V-ATPase dysfunction. Exemplary compounds of Formula (I) and salts, hydrates, deuterated analogs, and fluorinated analogs thereof are described in Section 5.2. Exemplary pharmaceutical compositions comprising compounds of Formula (I) and salts, hydrates, deuterated analogs, and fluorinated analogs thereof are described in Section 5.3. Exemplary features of methods of the disclosure are described in Section 5.4.

### 5.1. Definitions

Unless defined otherwise, all technical and scientific terms used herein have the same meaning as commonly understood to one of ordinary skill in the art to which this invention belongs. The following definitions are provided for the full understanding of terms used in this specification.

As used in the specification and embodiments, the singular form “a,” “an,” and “the” include plural references unless the context clearly dictates otherwise. For example, the term “an agent” includes a plurality of agents, including mixtures thereof.

Unless indicated otherwise, an “or” conjunction is intended to be used in its correct sense as a Boolean logical operator, encompassing both the selection of features in the alternative (A or B, where the selection of A is mutually exclusive from B) and the selection of features in conjunction (A or B, where both A and B are selected). In some places in the text, the term “and/or” is used for the same purpose, which shall not be construed to imply that “or” is used with reference to mutually exclusive alternatives.

The term V-ATPase refers to vacuolar-type ATPase. V-ATPases acidify the lumen of various organelles including lysosomes, endosomes, and secretory vesicles, and play a crucial role in the function of these organelles (Colacurio and Nixon, 2016, Ageing Rev. 2016 32:75-88). V-ATPases comprise a V₁ domain and a V₀ domain. Each domain includes multiple subunits, some of which have tissue specific isoforms. Genes encoding V₁ subunits include the ATP6V1A, ATP6V1B1, ATP6V1B2, ATP6V1C1, ATP6V1C2, ATP6V1D, ATP6V1E1, ATP6V1E2, ATP6V1F, ATP6V1G1, ATP6V1G2, ATP6V1G3, and ATP6V1H. Genes encoding V₀ subunits include the ATP6V0A1, ATP6V0A2, ATP6V0A3, ATP6V0A4, ATP6V0B, ATP6V0C, ATP6V0D1, ATP6V0D2, ATP6V0E1, and ATP6V0E2 genes.

The term pathogenic mutation refers to an alteration of a wild-type gene that is associated with a disease. A pathogenic mutation can be, for example, a point mutation in which a single nucleotide change results in a codon that codes for a different amino acid, a nonsense mutation that introduces a stop codon into the gene sequence, an insertion of one or more nucleotides, or a deletion of one or more nucleotides. The term wild-type refers to a gene sequence that predominates in a species, e.g., Homo sapiens.

A therapeutically effective amount of an agent or composition is an amount sufficient to achieve a desired therapeutic effect, and therefore does not require cure or complete remission.

The terms treat, treating, treatment, and grammatical variations thereof as used herein, include reducing or ameliorating a disorder or dysfunction, and/or signs or symptoms associated therewith, or slowing or halting the progression thereof. It will be appreciated that, although not precluded, treating a disorder or dysfunction does not require that the disorder, dysfunction or symptoms associated therewith be completely eliminated. Treatments according to the disclosure may be applied prophylactically (e.g., to a subject at risk of developing a disease or dysfunction associated with impaired lysosomal acidification), palliatively or remedially. Prophylactic treatments can be administered to a subject prior to onset of a sign or symptom, during early onset of a sign or symptom (e.g., upon initial signs and symptoms), or after an established development of a sign or symptom. Prophylactic administration can occur for several days to years prior to the manifestation of a symptom.

### 5.2. Compounds of Formula (I)

The methods of the disclosure comprise administering to a subject an amount of an agent which is a compound of Formula (I):

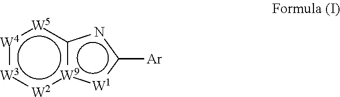

or a salt, hydrate, deuterated analog, or fluorinated analog thereof, wherein:
Ar is

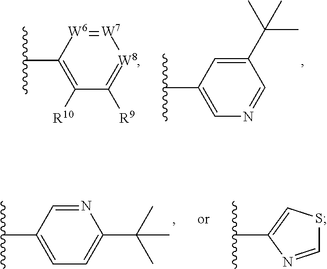

W¹ is N—R¹, O, or S, or when W° is N, W¹ may additionally be C—R⁵⁰;
W² is C—R² or N;
W³ is C—R³ or N;
W⁴ is C—R⁴ or N;
W⁵ is C—R⁵ or N;
W⁶ is C—R⁶ or N;
W⁷ is C—R⁷ or N;
W⁸ is C—R⁸ or N;
W⁹ is C, or when W¹ is C—R⁵⁰, W⁹ may be N;
R¹ is H, (C₁-C₃)alkyl, CH₂OC(═O)R³⁰, CH₂OP(═O)OR⁴⁰R⁴¹, C(═O)OR⁴², or C(═O)R⁴³;
each of R², R³, R⁴, and R⁵ is independently hydrogen, deuterium, halogen, perfluoro(C₁-C₄)alkyl, (C₁-C₄)alkyl, (C₁-C₄)alkoxy, perfluoro(C₁-C₄)alkoxy, (C₁-C₄)acyl, (C₁₋₄)alkoxy(C₁-C₄)alkyl, hydroxy(C₁-C₄)alkyl, hydroxy, carboxy, (C₁-C₄)alkoxycarbonylamino, carboxamido, (C₁-C₄)alkylaminocarbonyl, cyano, acetoxy, nitro, amino, (C₁-C₄)alkylamino, di(C₁-C₄)alkylamino, mercapto, (C₁-C₄)alkylthio, aminosulfonyl, (C₁-C₄)alkylsulfonyl, or (C₁-C₄)acylamino;
each of R⁶ and R¹⁰ is independently hydrogen, deuterium, halo, (C₁-C₃)alkyl, perfluoro(C₁-C₃)alkyl, hydroxy, (C₁-C₃)alkoxy, perfluoro(C₁-C₃)alkoxy, or amino;
each of R⁷ and R⁹ is independently hydrogen, deuterium, hydroxy, cyano, amino, halogen, halo(C₁-C₄)alkyl, (C₁-C₄)alkoxy, halo(C₁-C₄)alkoxy,

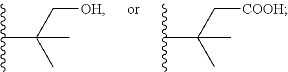

R⁸ is hydrogen, deuterium, halogen, halo(C₁-C₄)alkyl, (C₁-C₄)alkyl, (C₁-C₄)alkoxy, halo(C₁-C₄)alkoxy, cyano, phenyl, phenoxy, benzyloxy, amino,

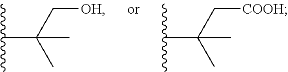

R³⁰ is (C₁-C₁₀)hydrocarbyl, (C₁-C₁₀)hydrocarbyl substituted with amino, (C₁-C₁₀)hydrocarbyl substituted with (C₁-C₄)hydrocarbyl, (C₁-C₁₀)hydrocarbyl substituted with carboxyl, carboxy, (C₁-C₆)alkoxycarbonyl, (C₁-C₆)alkoxycarbonylamino, methylthio, heterocyclyl, (C₁-C₁₀)oxaalkyl, CHR⁴⁴NHR⁴⁵ and guanidine;
each of R⁴⁰ and R⁴¹ is independently hydrogen or (C₁-C₆)hydrocarbyl;
R⁴² is (C₁-C₅)alkyl;
R⁴³ is (C₁-C₃)alkyl,
R⁴⁴ is a naturally occurring amino acid sidechain;
R⁴⁵ is H, methyl, or (C₁-C₄)alkoxycarbonyl; and
R⁵⁰ is H or (C₁-C₃)alkyl.

Compounds of Formula (I) are further described in PCT publication no. WO 2021/262617, the contents of which are incorporated herein by reference in their entireties.

Exemplary compounds of Formula (I) include the following compounds:

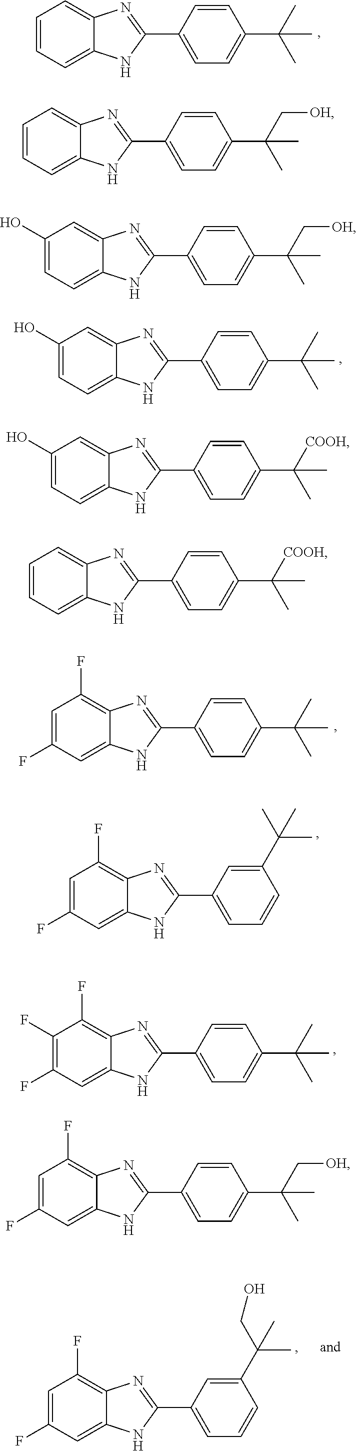

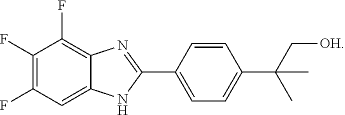 [SMILES: CC(C)(CO)c1ccc(-c2nc3c(F)c(F)c(F)cc3[nH]2)cc1]

In some embodiments, the agent is

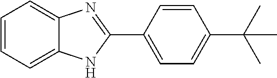 [SMILES: CC(C)(C)c1ccc(-c2nc3ccccc3[nH]2)cc1]

or a salt, hydrate, deuterated analog, or fluorinated analog thereof.

In some embodiments, the agent is

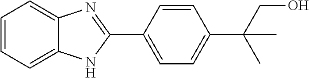 [SMILES: CC(C)(CO)c1ccc(-c2nc3ccccc3[nH]2)cc1]

or a salt, hydrate, deuterated analog, or fluorinated analog thereof.

In some embodiments, the agent is

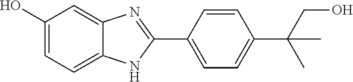 [SMILES: CC(C)(CO)c1ccc(-c2nc3cc(O)ccc3[nH]2)cc1]

or a salt, hydrate, deuterated analog, or fluorinated analog thereof.

In some embodiments, the agent is

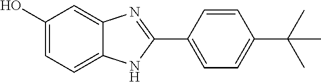 [SMILES: CC(C)(C)c1ccc(-c2nc3cc(O)ccc3[nH]2)cc1]

or a salt, hydrate, deuterated analog, or fluorinated analog thereof.

In some embodiments, the agent is

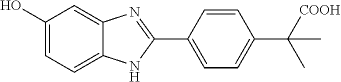 [SMILES: CC(C)(C(=O)O)c1ccc(-c2nc3cc(O)ccc3[nH]2)cc1]

or a salt, hydrate, deuterated analog, or fluorinated analog thereof.

In some embodiments, the agent is

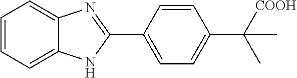 [SMILES: CC(C)(C(=O)O)c1ccc(-c2nc3ccccc3[nH]2)cc1]

or a salt, hydrate, deuterated analog, or fluorinated analog thereof.

In some embodiments, the agent is

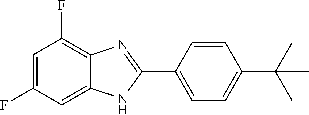 [SMILES: CC(C)(C)c1ccc(-c2nc3c(F)cc(F)cc3[nH]2)cc1]

or a salt, hydrate, deuterated analog, or fluorinated analog thereof.

In some embodiments, the agent is

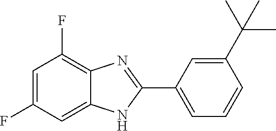 [SMILES: CC(C)(C)c1cccc(-c2nc3c(F)cc(F)cc3[nH]2)c1]

or a salt, hydrate, deuterated analog, or fluorinated analog thereof.

In some embodiments, the agent is

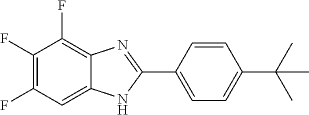 [SMILES: CC(C)(C)c1ccc(-c2nc3c(F)c(F)c(F)cc3[nH]2)cc1]

or a salt, hydrate, deuterated analog, or fluorinated analog thereof.

In some embodiments, the agent is

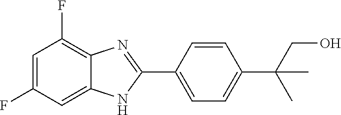 [SMILES: CC(C)(CO)c1ccc(-c2nc3c(F)cc(F)cc3[nH]2)cc1]

or a salt, hydrate, deuterated analog, or fluorinated analog thereof.

In some embodiments, the agent is

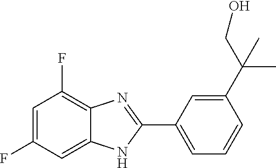 [SMILES: CC(C)(CO)c1cccc(-c2nc3c(F)cc(F)cc3[nH]2)c1]

or a salt, hydrate, deuterated analog, or fluorinated analog thereof.

In some embodiments, the agent is

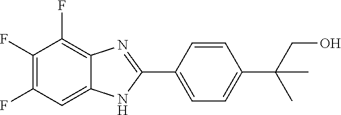 [SMILES: CC(C)(CO)c1ccc(-c2nc3c(F)c(F)c(F)cc3[nH]2)cc1]

or a salt, hydrate, deuterated analog, or fluorinated analog thereof.

In some embodiments, the agent is a compound of Formula (I) (e.g., one of the specific compounds of Formula (I) whose structure is shown in this Section) or a salt thereof.

In some embodiments, the agent is ZLN-005. In other embodiments, the agent is a salt of ZLN-005.

### 5.3. Pharmaceutical Compositions

Compounds of Formula (I) and salts, hydrates, deuterated analogs, and fluorinated analogs thereof can be formulated for the intended route of administration, for example according to techniques known in the art (e.g., as described in Allen et al., eds., 2012, Remington: The Science and Practice of Pharmacy, 22^{nd} Edition, Pharmaceutical Press, London, UK). Suitable routes of administration include, but are not limited to, intravenous and oral routes of administration. Suitable routes also include pulmonary administration, including by inhalation. The most suitable route may depend upon the condition or disorder of the subject.

Compounds of Formula (I) and salts, hydrates, deuterated analogs, and fluorinated analogs thereof can be formulated as a pharmaceutical composition comprising a compound of Formula (I), or a salt, hydrate, deuterated analog, or fluorinated analog thereof and one or more pharmaceutical excipients, for example one or more excipients described in Handbook of Pharmaceutical Excipients, 8^{th} Revised Ed. (2017), incorporated by reference in its entirety. The pharmaceutical compositions can be presented in unit dosage form.

### 5.4. Uses of Compounds of Formula (I)

The disclosure provides methods of treating subjects with compounds of Formula (I) (e.g., ZLN-005) and salts, hydrates, deuterated analogs, and fluorinated analog thereof. In the methods of the disclosure, the subjects are preferably mammals (e.g., primates or rodents such as mice or rats), most preferably humans.

In one aspect, the disclosure provides a method of treating a subject having a disease or disorder associated with impaired lysosomal acidification comprising administering to the subject a therapeutically effective amount of an agent, where the agent is a compound of Formula (I) (e.g., ZLN-005) or a salt, hydrate, deuterated analog, or fluorinated analog thereof. A therapeutically effective amount of the agent can be, for example, an amount that is effective to increase lysosomal acidity in the subject. Diseases and disorders associated with impaired lysosomal acidification include sepsis (for example in subjects with bowel perforations and/or an abdominal cavity infection), infections (including bacterial infections, e.g., multi-drug resistant bacterial infections, fungal infections, parasitic infections, and viral infections), and V-ATPase dysfunction associated diseases and disorders such as renal tubular acidosis, Zimmerman n-Laband syndrome, Cutis laxa type II or wrinkly skin syndrome, osteopetrosis, glucose intolerance, diabetes, Parkinson's disease, Alzheimer's disease, and hearing loss.

In another aspect, the disclosure provides a method for increasing lysosomal acidity in a subject comprising administering to the subject an amount of an agent effective to increase lysosomal acidity in the subject, where the agent is a compound of Formula (I) (e.g., ZLN-005) or a salt, hydrate, deuterated analog, or fluorinated analog thereof.

In another aspect, the disclosure provides a method of treating a subject having sepsis (particularly subjects not having systemic immune activation), an infection, or a disease associated with V-ATPase dysfunction, comprising administering to the subject a therapeutically effective amount of an agent, wherein the agent is a compound of Formula (I) (e.g., ZLN-005) or a salt, hydrate, deuterated analog, or fluorinated analog thereof. In some embodiments, the subject has polymicrobial sepsis (e.g., caused by a combination of two or more bacteria, a combination of two or more fungi, or combination of one or more bacteria and one or more fungi). In other embodiments, the subject has unimicrobial sepsis (e.g., caused by a single species of pathogen such as a bacterium or fungus). In some embodiments, the subject has early sepsis. Common (though not necessarily present) indicators of early sepsis include a fever (e.g., above 38° C.), a heart rate above 90 beats per minute, an infection confirmed through a positive blood culture, and a rapid breathing rate of more than 20 breaths per minute.

In some embodiments, where a subject has an infection (e.g., bacterial, fungal, viral, parasitic, or a combination thereof), administration of the agent can lead to a reduction in pathogen load. For example, administration of the agent to a subject having a bacterial infection can reduce the subject's bacterial load. Methods for assessing pathogen (e.g., bacterial) load levels are known in the art. See, e.g., Stranieri et al., 2018, Rev Inst Med Trop Sao Paulo. 60:e61.

A subject, e.g., a subject having an infection or sepsis can be assessed using the Sequential Organ Failure Assessment (SOFA) (Vincent et al., 1996, Intensive Care Med 22(7):707-10). SOFA scores range from 0 to 24, with higher scores correlating to higher mortality. In some embodiments, a subject treated according to a method of the disclosure has a SOFA score of 0 to 6 (e.g., 0 to 3, 1 to 4, 2 to 5, 0, 1, 2, 3, 4, 5, or 6) prior to treatment with an agent of the disclosure (e.g., ZLN-005). In some embodiments, a subject has a SOFA score of 0 to 2 prior to treatment with an agent of the disclosure (e.g., ZLN-005). In some embodiments, a subject has a SOFA score of 2 to 6 prior to treatment with an agent of the disclosure (e.g., ZLN-005) In some embodiments, a subject treated according to a method of the disclosure has a SOFA score of 7 to 9 (e.g., 7, 8, or 9) prior to treatment with an agent of the disclosure (e.g., ZLN-005). In some embodiments, a subject treated according to a method of the disclosure has a SOFA score of 10 to 12 (e.g., 10, 11, or 12) prior to treatment with an agent of the disclosure (e.g., ZLN-005). In some embodiments, a subject treated according to a method of the disclosure has a SOFA score of 13 to 14 (e.g., 13 or 14) prior to treatment with an agent of the disclosure (e.g., ZLN-005). In some embodiments, a subject treated according to a method of the disclosure has a SOFA score of 15 to 24 (e.g., 15 to 20, 20 to 24, 15, 16, 17, 18, 19, 20, 21, 22, 23, or 24) prior to treatment with an agent of the disclosure (e.g., ZLN-005).

In some embodiments, a subject has an infection with at least one species of pathogen and one, two, three, or all four of the following: (1) lower than normal levels of HLA-DR expression in the peripheral blood; (2) higher than normal PD-1 expression in T cells; (3) lower than normal CD88 expression in neutrophils, and (4) lower than normal Thi7/Treg ratio. HLA-DR strongly correlates with immune system cell activation, and HLA-DR expression in the peripheral blood of patients with sepsis has been reported to be reduced compared to healthy individuals Winkler et al., 2017, PLoS One 12:e0182427. In addition, a frequency of HLA-DR-expressing mononuclear cells below 30% is reported to be immunosuppressed (Misra et al., 2020, Crit Care Clin 36:167-176). Accordingly, in some embodiments, a subject has a frequency of HLA-DR-expressing mononuclear cells below 30%. In T cells, enhanced expression of inhibitory signals such as program cell death 1 (PD-1) correlates with poor prognosis in septic patients (Boomer et al., 2012, Crit Care 16:R112). Immune checkpoint inhibitors used in the treatment of cancer have also had a significant effect in the treatment of sepsis models (Huang et al., 2022, Mol Ther 30:1227-1238). This demonstrated a major role of Tregs in the immunosuppressive state of sepsis. In addition, the Th17/Treg ratio was found to be a good indicator of the state of the immune system (Gupta et al., 2016, Cytokine 88:214-221). In the typical course of sepsis, this ratio rises in the eariy stages of sepsis and then declines, with the decline suggesting an immunosuppressive state, in line with the decline in HLA-DR (Xu et al., 2020, Scand J Immunol 91:e12813). In neutrophils, activated complement C₅a inhibits RhoA polymerization, rendering it inactive, and resulted in reduced CD88 expression, which has a function in phagocytosis (Morris et al., 2011, Blood 117:5178-5188). Weakened CD88 expression has been reported to reflect well the immunosuppressive state and strongly correlate with secondary infection (Conway et al., 2018, Intensive Care Med 44:627-635).

In the methods of the disclosure, the amount of the agent can be an amount effective to increase lysosome acidity in phagocytic cells of the subject (e.g., macrophages, monocytes, or neutrophils). Lysosomal acidity can be measured by lysosomal pH detection assays, for example the Dojindo Lysosomal Acidic pH Detection Kit (Dojindo product code L266).

In the methods of the disclosure, the amount of the agent can be an amount effective to increase lysosome acidity in peritoneal cells of the subject, for example peritoneal phagocytic cells (e.g., macrophages, monocytes, or neutrophils).

In the methods of the disclosure, the amount of the agent can be an amount effective to increase Tfeb (transcription factor EB) mRNA levels in phagocytic cells of the subject (e.g., macrophages, monocytes, or neutrophils). In the methods of the disclosure, the amount of the agent can be an amount effective to increase TFEB activation (dephosphorylation) in phagocytic cells of the subject (e.g., macrophages, monocytes, or neutrophils). In the methods of the disclosure, the amount of the agent can be an amount effective to increase TFEB nuclear migration in phagocytic cells of the subject (e.g., macrophages, monocytes, or neutrophils).

In the methods of the disclosure, the amount of the agent can be an amount effective to increase phosphorylated Akt (protein kinase B) to Akt (p-Akt/Akt) ratio in phagocytic cells of the subject (e.g., macrophages, monocytes, or neutrophils).

In the methods of the disclosure, the amount of the agent can be an amount effective to increase phosphorylated PI3K (phosphoinositide 3-kinase) to PI3K (p-PI3K/PI3K) ratio in phagocytic cells of the subject (e.g., macrophages, monocytes, or neutrophils).

In the methods of the disclosure, the amount of the agent can be an amount effective to increase physical contact between mitochondia and lysosomes in phagocytic cells of the subject (e.g., macrophages, monocytes, or neutrophils).

In the methods of the disclosure, the amount of the agent can be an amount effective to increase spare respiratory capacity in phagocytic cells of the subject (e.g., macrophages, monocytes, or neutrophils).

In the methods of the disclosure, the amount of the agent can be an amount effective to increase glycolytic capacity in phagocytic cells of the subject (e.g., macrophages, monocytes, or neutrophils).

In the methods of the disclosure, the amount of the agent can be an amount effective to increase glycolytic reserve in phagocytic cells of the subject (e.g., macrophages, monocytes, or neutrophils).

In the methods of the disclosure, the amount of the agent can be an amount effective to increase lysosomal proteolysis in phagocytic cells of the subject (e.g., macrophages, monocytes, or neutrophils).

In the methods of the disclosure, the amount of the agent can be an amount effective to increase mRNA levels of hydrolases (e.g., Ctsd (cathepsin D) and/or membrane proteins (e.g., Atp6v1A, Atp6v0d1, or Mcoln1 (mucolipin-1)) in phagocytic cells of the subject (e.g., macrophages, monocytes, or neutrophils).

In the methods of the disclosure, the amount of the agent can be an amount effective to reduce the level of one or more markers of inflammation (e.g., in blood or serum). For example, the one or more markers of inflammation can comprise Tnfα, IL1β, IL6, IFNγ, or a combination thereof.

In some embodiments of the methods described herein, treating can comprise reducing or alleviating one or more symptoms of a disease or dysfunction experienced by the subject.

In some embodiments of the methods of the disclosure, the subject does not have systematic immune activation. Systemic immune activation can be associated with cytokine release syndrome (CRS). Accordingly, in some embodiments, the subject does not have CRS. Systemic immune activation and CRS are associated with elevated cytokines, including interleukin-6 (IL-6), interleukin-10 (IL-10), interferon (IFN)-γ, monocyte chemoattractant protein 1 (MCP-1), granulocyte-macrophage colony-stimulating factor (GM-CSF), tumor necrosis factor (TNF), IL-1, IL-2, IL-2-receptor-α, IL-8, IL-17A, and IL-17F (see, e.g., Murthy et al., 2019 Immunotargets Ther. 8:43-52). Elevated C-reactive protein (CRP) is also observed in CRS. In some embodiments, the subject does not have elevated IL-6. In some embodiments, the subject does not have elevated IL-10. In some embodiments, the subject does not have elevated IFN-γ. In some embodiments, the subject does not have elevated MCP-1. In some embodiments, the subject does not have elevated GM-CSF. In some embodiments, the subject does not have elevated TNF. In some embodiments, the subject does not have elevated IL-1. In some embodiments, the subject does not have elevated IL-2. In some embodiments, the subject does not have elevated IL-2-receptor-α. In some embodiments, the subject does not have elevated IL-8. the subject does not have elevated IL-2. In some embodiments, the subject does not have elevated IL-17A. the subject does not have elevated IL-2. In some embodiments, the subject does not have elevated IL-17-F. In some embodiments, the subject does not have elevated CRP. In some embodiments, the subject does not have an elevated D-dimer level.

Levels of biomarkers such as those described in this paragraph can be measured by standard laboratory assays. Biomarker levels can be considered elevated when a measured value is above the upper limit of normal.

In some embodiments, the subject's pre-treatment serum IL-6 level is less than 200 pg/ml, 150 pg/ml, 100 pg/ml, 90 pg/ml, 80 pg/ml, 70 pg/ml, 60 pg/ml, 50 pg/ml, 40 pg/ml, 30 pg/ml, 20 pg/ml, 10 pg/ml, 5 pg/ml, 4 pg/ml, 3 pg/ml, or 2.5 pg/ml. In some embodiments, the subject's pre-treatment serum CRP level is less than 40 mg/L, 35 mg/L, 25 mg/L, 20 mg/L, 20 mg/L, 15 mg/L, 10 mg/L, 5 mg/L, or 2 mg/L.

In some embodiments of the methods of the disclosure, the subject has suppressed innate immune function. Innate immune function can be measured by assaying the phagocytic ability of a subject's phagocytes (e.g., macrophages). A subject's innate immune function can be considered suppressed when the phagocytic ability of the subject's phagocytes is lower than the phagocytic ability of phagocytes from healthy subjects. Phagocytosis activity assay kits are commercially available. An exemplary phagocytosis assay kit is the Cayman Chemical Phagocytosis Assay Kit (Cayman item no. 500290). In some embodiments, the amount of the agent administered is an amount effective to enhance the subject's innate immune function (e.g., as measured by phagocytic activity of phagocytes such as macrophages).

In some embodiments of the methods of the disclosure, the subject has sepsis (e.g., polymicrobial sepsis or unimicrobial sepsis) and (i) has suppressed innate immune function, (ii) does not have systemic immune activation, or (iii) has suppressed innate immune function and does not have systemic immune activation. In some embodiments, the subject has a bowel perforation.

In some embodiments of the methods of the disclosure, the subject has a bacterial infection, for example, an infection caused by a multi-drug resistance bacterium. Exemplary multi-drug resistant bacteria include vancomycin-resistant Enterococci (VRE), methicillin-resistant Staphylococcus aureus (MRSA), extended-spectrum P-lactamase (ESBL) producing gram-negative bacteria, Klebsiella pneumoniae carbapenemase (KPC) producing gram-negative bacterial, and multidrug-resistant gram negative (MDRGN) bacteria such as Enterobacter species, E. coli, Klebsiella pneumoniae, Acinetobacter baumannii, and Pseudomonas aeruginosa. In other embodiments, the subject has a fungal infection (e.g., a Candida albicans infection). In other embodiments, the subject has a parasitic infection (e.g., a Plasmodium infection, which causes malaria, or a Trypanosoma cruzi infection, which causes Chagas disease). In other embodiments, the subject has a viral infection (e.g., influenza). In some embodiments, the subject has an infection of the abdominal cavity

In some embodiments of the methods of the disclosure, the subject has a V-ATPase dysfunction (e.g., caused by a pathogenic mutation in a gene encoding a V-ATPase subunit or V-ATPase accessory protein) and/or a disease or disorder associated with a V-ATPase dysfunction. Exemplary diseases and disorders associated with V-ATPase dysfunction include example renal tubular acidosis, Zimmermann-Laband syndrome, Cutis laxa type II or wrinkly skin syndrome, osteopetrosis, glucose intolerance, diabetes, Parkinson's disease, Alzheimer's disease, and hearing loss. Pathogenic ATP6V1B1 and ATP6V0A4 mutations can cause renal tubular acidosis (Uzak et al., 2013, Ren. Fail. 35(9):1281-1284; Stover et al., 2002, J. Med. Genet. 39(11):796-803). Pathogenic ATP6V1B2 mutations can cause Zimmermann-Laband syndrome (Kortum et al., 2015, Nat. Genet. 47(6):661-7). Pathogenic ATP6V0A2 mutations can cause cutis laxa type II or wrinkly skin syndrome (Kornak et al., 2008, Nat Genet. 40(1):32-4). Pathogenic ATP6V0A3 mutations can cause osteopetrosis, which can be additionally associated with neurological complications (Bhargava et al., 2012, JBC 287(32):26829-26839; Steward, 2003, Neuropathol Appl Neurobiol. 29(2):87-97). Deficiency of ATP6V1H is associated with impaired glucose tolerance and diabetes (Yang et al., 2022, Arch Biochem Biophys. 716:109116). Pathogenic mutations in ATP6AP2 (which encodes a V-ATPase accessory protein) can cause Parkinson's disease, e.g., X-linked Parkinson Disease with Spacticity (XPDS) (Korvatska et al., 2013, Hum Mol Genet. 22(16):3259-88). Pathogenic mutations in PSEN1 (which encodes presenilin-1, a protein involved in V-ATPase targeting to lysosomes) is associated with Alzheimer's disease (Lee et al., 2015, Cell Rep. 12(9):1430-1444). Pathogenic mutations in DMXL2 (which encodes Dmx-like 2, a protein involved in modulating V-ATPase activity) are associated with hearing loss (Chen et al., 2017, Genetics in Medicine 19:553-558).

In some embodiments of the methods of the disclosure, the subject does not have a neurodegenerative disorder.

In the methods of the disclosure, the agent can be administered by any suitable means, for example enterically. In some embodiments, the agent is administered by mouth.

In some embodiments of the methods of the disclosure, agent is administered in a dose ranging from 0.5 mg/kg to 1000 mg/kg per day. In some embodiments, the dose is 25 mg/kg to 1000 mg/kg per day.

### 6. EXAMPLES

### 6.1. Example 1: ZLN-005 Rescue of Septic Animals in the Cecal Ligation perforation model

A study was performed to evaluate the ability of ZLN-005 to rescue mice from sepsis in the cecal ligation and perforation (CLP) model.

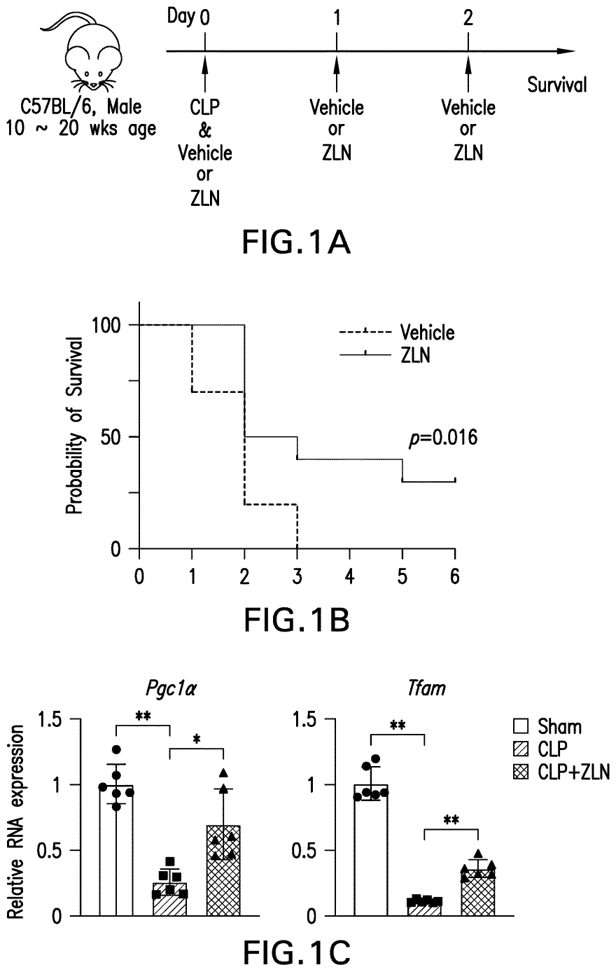

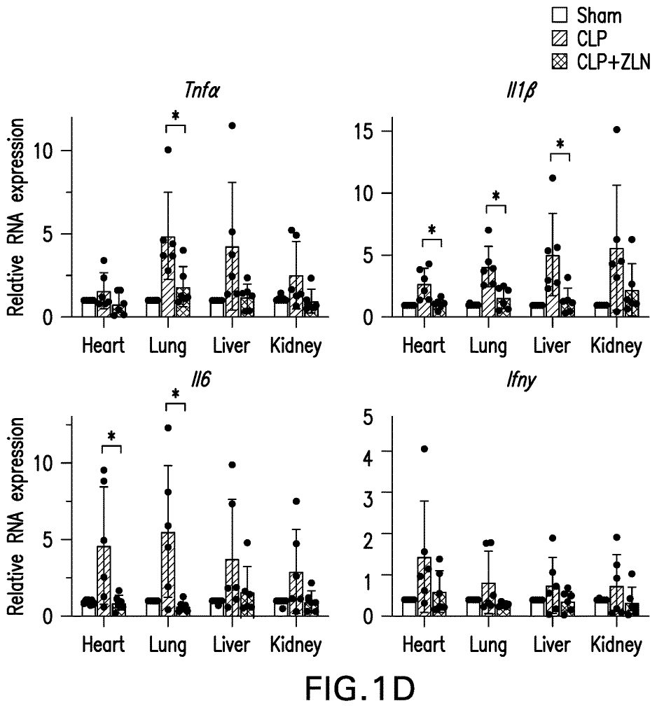

Cecal ligation and perforation was performed on C57BL/6 mice about 10 to 20 weeks of age. Mice were administered either ZLN-005 at 12 mg/kg (i.p.) or vehicle (DMSO) at the time of cecal ligation and perforation, 24 hours later, and 48 hours later (FIG. 1A).

Survival proportions for mice administered either ZLN-005 or vehicle are shown in FIG. 1B. ZLN-005 increased the probability of survival compared to vehicle.

### 6.2. Example 2: ZLN-005 Increases Phagocytic Activity of Macrophages In Vitro

A study was performed to evaluate the ability of ZLN-005 to enhance the phagocytic activity of macrophages.

THP-1 cells were differentiated to macrophages by incubating the cells with 10 nM phorbol 12-myristate 13-acetate (PMA) for 48 hours (study hours 0 to 48). Cells were then incubated with 1 μM ZLN-005 or vehicle (DMSO) for five days (study hours 48 to 120), with or without LPS at 1 μg/mL for the final day (study hours 96 to 120). Cells were then incubated with latex beads for 20 minutes and assayed for phagocytic activity by the Phagocytosis Assay Kit (IgG FITC) (Cayman Chemical, item no. 500290).

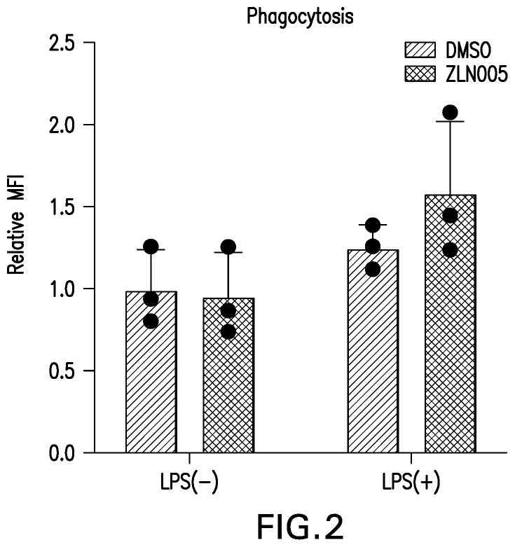

Results are shown in FIG. 2. ZLN-005 treatment increased phagocytic activity of LPS-stimulated macrophages (see, FIG. 2, right bar).

### 6.3. Example 3: ZLN-005 Increases Lysosomal Acidity of Macrophages In Vitro

A study was performed to evaluate the ability of ZLN-005 to increase lysosomal acidity of macrophages.

THP-1 cells were differentiated to macrophages by incubating the cells with 10 nM phorbol 12-myristate 13-acetate (PMA) for 48 hours (study hours 0 to 48). Cells were then incubated with 1 μM ZLN-005 or vehicle (DMSO) for five days (study hours 48 to 120), with or without LPS at 1 μg/mL for the final day (study hours 96 to 120). Cells were then stained for lysosomal acidity using a pH sensitive dye.

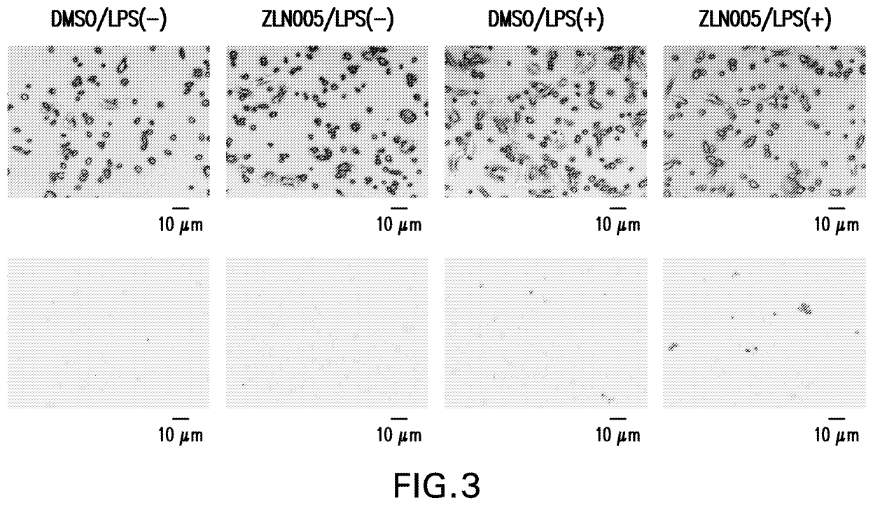

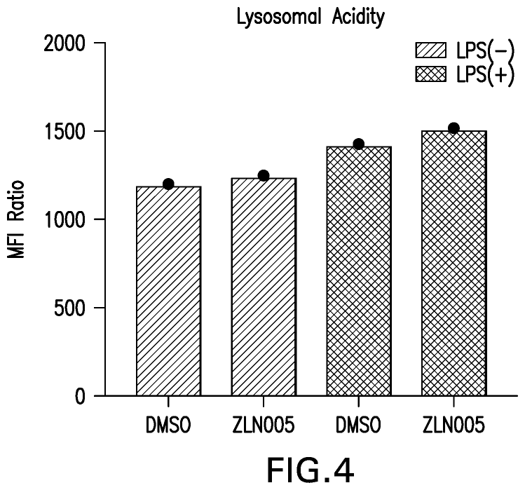

Microscopy images of stained cells are shown in FIG. 3. The pH sensitive dye permeates lysosomes based on pH, with fluorescence intensity increasing as acidity increases. ZLN-005 treatment was found to enhance lysosomal acidity of LPS stimulated cells (see, FIG. 3, bottom right image). MFI ratios for the different treatment groups are shown in FIG. 4.

### 6.4. Example 4: ZLN-005 Increases Phagocytic Activity and Lysosomal Acidity in Vivo

A study was performed to evaluate the ability of ZLN-005 to increase the phagocytic activity of phagocytes in vivo.

Cecal ligation and perforation was performed on mice at study hour 0. Mice were administered either ZLN-005 at 12 mg/kg (i.p.) or vehicle (DMSO) at the time of cecal ligation and perforation. Peritoneal cells were collected 24 hours later by peritoneal lavage and assayed for phagocytic activity and lysosomal acidity. Peritoneal bacteria levels were also measured.

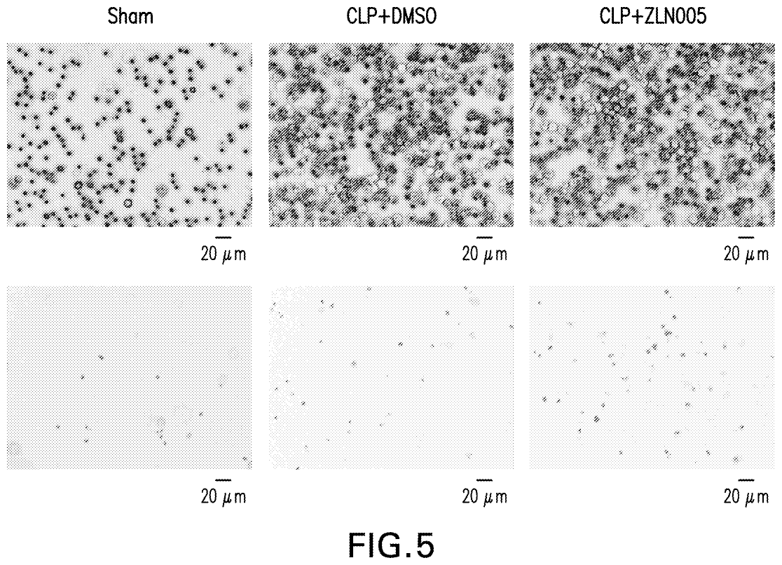

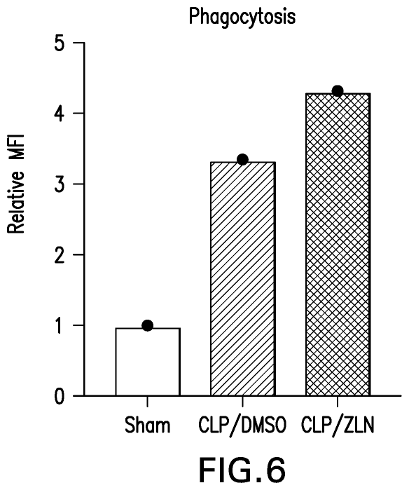

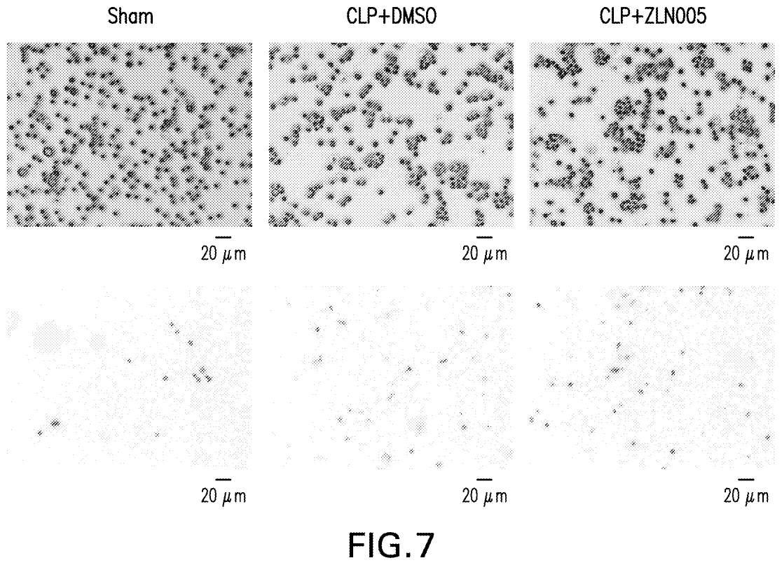

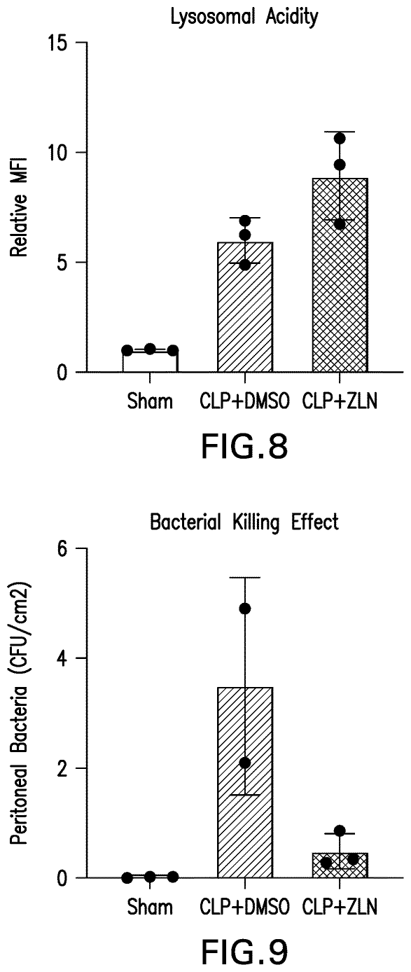

Results are shown in FIGS. 5-9. Mice treated with ZLN-005 were found to have phagocytic cells with increased phagocytic activity (FIGS. 5-6), lysosomal pH (FIGS. 7-8), and bacterial killing ability (FIG. 9) compared to mice treated with vehicle (DMSO).

### 6.5. Example 5: ZLN-005 Improves Sepsis Survival

This Example describes studies of ZLN-005 relating to sepsis. Some of the studies described in this Example correspond to studies described in the foregoing Examples, but this Example includes additional details relating to the materials and methods and more detailed analysis of study results.

### 6.5.1. Materials and Methods

### 6.5.1.1. Sepsis Model Mice

C57BL/6 mice were purchased from Shimizu Laboratory Supplies Co., Ltd. (Kyoto, Japan). Mice were housed in specific-pathogen-free conditions with free access to food and water. Mice were anesthetized by inhalation of isoflurane (099-06571, FUJIFILM Wako Pure Chemical Corporation, Tokyo, Japan). A midline incision was made, followed by externalization, and the cecum was then ligated at half the distance between the distal pole and the base of the cecum and punctured with a 21-gauge needle (NN-2116R, Terumo Corporation, Tokyo, Japan). Next, a small amount of fecal mass from the punctured cecum was gently squeezed out to ensure patency of punctures, the cecum was relocated, and 6/0 Ethicon PROLENE Suture (Ethicon, Inc., Raritan, NJ, USA) was used to close the peritoneum and skin. Sham-operated mice underwent only incision and cecum exteriorization.

### 6.5.1.1. Intraperitoneal Injection of ZLN-005

ZLN-005 (S7447, Selleck Chemicals, Houston, TX, USA) stock solutions were prepared in dimethyl sulfoxide (DMSO) (048-21981, FUJIFILM Wako Pure Chemical Corporation) adjusted to a 10 mM concentration. Mice were injected with ZLN-005 (12 mg/kg) or the same amount of DMSO as the control group every day from Day 0 to Day 2. Mice were examined continuously for survival until 6 days post-CLP.

### 6.5.1.2. Isolation of Mouse Peritoneal Cavity Cells

The outer skin of the peritoneum was cut with scissors and gently pulled back to expose the inner skin lining the peritoneal cavity. The inner skin was punctured with an 18G Surflo I.V. Catheter (SR-FS1851, Terumo Corporation, Tokyo), and 4 ml of ice-cold PBS was injected. After injection, the abdomen was gently massaged to dislodge peritoneal cavity cells. The collected fluid was filtered through a Falcon 40-μm Cell Strainer (352340, Corning Inc., Corning, NY, USA) and centrifuged at 800×g for 5 minutes. Cell pellets were resuspended in 5 ml of 1× Lysing Buffer (555899, Becton, Dickinson and Company, Franklin Lakes, NJ, USA) and incubated for 5 minutes at room temperature to lyse erythrocytes. Then, 10 ml of PBS was added and centrifuged at 800×g for 5 minutes, the supernatant was discarded, and the cells were resuspended in PBS or culture medium.

### 6.5.1.3. Population Analysis of Peritoneal Cavity Cells

Peritoneal cavity cells were harvested 24 hours after CLP. The cells were stained with a PE anti-mouse/human CD11b antibody (101207, BioLegend, Inc., San Diego, Califomia, USA) and FITC anti-mouse F4/80 antibody (123108, BioLegend, Inc.) or PE Rat IgG2b, κ isotype control antibody (400608, BioLegend, Inc.) and FITC Rat IgG2a, κ isotype control antibody (400505, BioLegend, Inc.) for 30 minutes at 4° C. after blocking the nonspecific Fc receptor using the Fc blocking reagent (130-059-901, Miltenyi Biotec, Bergisch Gladbach, Germany) for 10 minutes at room temperature. After staining, the cells were washed immediately and resuspended in AutoMACS® Running Buffer (130-091-221, Miltenyi Biotec). Fluorescence data were collected using a SH800 cell sorter (Sony Biotechnology Inc., Tokyo, Japan). The flow cytometry files were analyzed using FlowJo™ software (Ver. 10.8.1, Becton, Dickinson and Company).

### 6.5.1.4. Echocardiographic Measurement

Chest hair was removed with cream one day before echocardiography was performed using VisualSonics Vevo®@2100 equipped with an 18- to 38-MHz probe (VisualSonics, Toronto, ON, Canada). Mice were anesthetized by inhalation of isoflurane at 24 hours post-CLP. The left ventricle was assessed in the parasternal short-axis view. Left ventricular end-systole or end-diastole was defined as the period when the left ventricular lumen was shortest or most dilated, respectively. Diastolic left ventricular (LV) internal diameters, systolic LV internal diameters, diastolic LV anterior wall, and diastolic LV posterior wall were measured from the LV M-mode tracing at the papillary muscle level. LV wall thickness was calculated as the average of anterior and posterior wall thicknesses.

### 6.5.1.5. Histology and Inflammatory Scores

A study was performed to evaluate the ability of ZLN-005 to rescue mice from sepsis in the cecal ligation and perforation (CLP) model.

The heart, lung, liver, right kidney, and spleen of mice were collected 24 hours post-CLP and fixed with 4% paraformaldehyde (163-20145, FUJIFILM Wako Pure Chemical Corporation). All tissues were embedded in paraffin, cut into sections, and stained with hematoxylin and eosin (HE). Liver inflammatory scores were assessed based on the severity of necrosis, bleeding, and infiltration in the liver using the method described in Shikuma et al., 2022 Front Immunol 13:825171. Lung inflammatory scores were assessed based on the severity of edema, intra-alveolar cell infiltration, congestion and alveolar hemorrhage using the method described in An et al., 2019, Sci Rep 9:2836.

### 6.5.1.6. Cell Culture

THP-1 cells of a human monocytic leukemia cell line were cultured in Roswell Park Memorial Institute 1640 medium (11875-093, RPM11640, Thermo Fisher Scientific Inc., Waltham, MA, USA) supplemented with 10% fetal bovine serum (FBS, 10270-106, Thermo Fisher Scientific Inc.). Cells were incubated at 37° C. in a humidified 5% CO₂ incubator. For the THP-1 inflammation model, the cells were seeded in 12-well cell culture plates (353043, Corning Inc.) at a density of 5×10⁵ cells per well in growth medium containing 10 nM phorbol 12-myristate 13-acetate (PMA, AG-CN2-0010-M001, Adipogen Life Sciences Inc., San Diego, CA, USA). After 48 hours, the supernatant of the medium was carefully removed to avoid detaching the cells attached to the bottom of the plate and replaced with new medium containing 1 μM ZLN-005 or DMSO (0.1%) as a control. Forty-eight hours later, 1 μg/ml LPS (lipopolysaccharides, 125-05181, FUJIFILM Wako Pure Chemical Corporation) was added, and the cells were harvested at each time point and used for studies. For PI3K inhibition, the cells were seeded in the plate and added 200 nM Wortmannin (AG-CN2-0023-M001, Adipogen Life Sciences Inc.). For PGC1α gene knock down, cells were nucleofected with 10 nmole siRNA-PGC1α (4427037, Thermo Fisher Scientific Inc.) and 10 nmole siRNA-negative control (4390844, Thermo Fisher Scientific Inc.) using the Nucleofector™ 2b (Lonza, Walkersvill, MD, USA) according to the manufacturer's protocol. For autophagy flux analysis, cells were stimulated with 1 μg/ml LPS for 6 hours, followed by 50 nM bafilomycin A1 (B0025, LKT Laboratories, Inc., Saint Paul, MN, USA) or 30 nM chloroquine (08660-04, Nacalai Tesque Inc., Kyoto, Japan) for 2 or 6 hours.

### 6.5.1.7. RNA Isolation, Reverse Transcription PCR and Quantitative PCR

Total RNA from cells and tissues was extracted using TRIzol™ (15596018, Thermo Fisher Scientific Inc.) and a Direct-zol™ RNA MiniPrep Kit (R2052, Zymo Research, Irvine, CA, USA) with DNase 1, according to the manufacturer's recommendations. To perform the qRT-PCR assay, 100 ng of total RNA was reverse-transcribed using the PrimeScript™ RT Reagent Kit (RR036A, Takara Bio, Shiga, Japan) and a T100 thermal cycler (Bio-Rad Laboratories, Inc.). qRT-PCR was performed with Kapa SYBR® Fast qPCR Kit Master Mix (2×) Universal (KK4602, Kapa Biosystems Ltd., Wilmington, MA, USA) on a CFX Connect™ m real-time system (Bio-Rad Laboratories, Inc.). The relative gene expression levels were normalized to GAPDH (or Gapdh) expression. The mtDNA copy number (CN) was estimated from the content ratio of 12S rRNA on mtDNA and ACTB (or Actb) on nuclear DNA by delta cycle threshold-based relative quantification.

### 6.5.1.8. Mitochondrial Membrane Potential (Δφ)

Cells were resuspended at a density of 1×10⁵/mi in culture medium containing 100 nM MitoTracker™ Green FM (MitoG, Thermo, M7514, Fisher Scientific, Inc.) and 100 nM Image-iT™ TMRM Reagent (TMRM, T668, Thermo Fisher Scientific, Inc.) and incubated at 37° C. for 30 minutes. After staining, the cells were washed immediately, resuspended in AutoMACS® running buffer, and evaluated using an SH800 cell sorter. The fluorescence intensity was analyzed by FlowJo™, and the numeric value was calculated by dividing the fluorescence intensity of TMRM by the fluorescence intensity of MitoG (TMRM/MitoG) as an index of Δφ.

6.5.1.9. Measurement of Mitochondrial Reactive Oxygen Species (mtROS) Levels

Cells were resuspended at a density of 1×10⁵/mi in culture medium containing 5 μM MitoSOX™ Red mitochondrial superoxide indicator (MitoSOX, M36008, Thermo Fisher Scientific, Inc.) and incubated at 37° C. for 30 minutes. After staining, the cells were washed immediately, resuspended in AutoMACS® running buffer, and evaluated using an MA900 cell sorter (Sony Biotechnology Inc., Tokyo, Japan).

### 6.5.1.1. Measurement of Cellular Reactive Oxygen Species (ROS)

Cells were resuspended at a density of 1×10⁵/mi in culture medium containing 5 μM CellROX™ Deep Red (CellROX, C10491, Thermo Fisher Scientific, Inc.) and incubated at 37° C. for 30 minutes. After staining, the cells were washed immediately, resuspended in AutoMACS® running buffer, and evaluated using an SH800 cell sorter.

### 6.5.1.2. Mitophagy Detection Assay

To detect mitophagy, the pMX retroviral vector carrying Monomeric Keima Red (mKeima Red) was transfected into THP-1 cells. The mKeima Red-expressing cells were selected using SH800 cell sorter one week after retrovirus transfection. One more sorting was performed to obtain over 95% cells expressing mKeima Red. The acidic mKeima Red signal was detected by an Attune® NxT Flow Cytometer (Thermo Fisher Scientific). mKeima Red was set at 488-nm (pH 7) and 561-nm (<pH 6) lasers with 590/40-nm and 615/20-nm emission filters, respectively. The mitophagy index was defined as the ratio of acidic (<pH 6) mKeima Red signal-positive cells to DMSO control cells at 0 hours (Suzuki et al., 2017, Biochemical and Biophysical Research Communications 483:88-93).

### 6.5.1.3. Phagocytosis Assay

Phagocytosis was assessed using a Phagocytosis Assay Kit IgG-FITC (500290, Cayman Chemical, Ann Arbor, MI, USA). Cells were suspended at a concentration of 3×10⁵ in 1 ml of culture medium and stained with the Latex Beads-rabbit IgG-FITC Complex from the kit for 20 minutes at 37° C. After staining, the cells were centrifuged at 400×g for 5 minutes and resuspended in 200 μl of autoMACS® running buffer, and evaluated using an SH800 cell sorter.

### 6.5.1.4. Lysosomal Acidification

Lysosomal acidification was assessed using pHrodo™ Green dextran (P35388, Thermo Fisher Scientific, Inc.). Peritoneal cavity cells suspended at a concentration of 3×10⁵ in 1 ml of culture medium were stained with 50 μg/ml pHrodo™ Green dextran from the kit for 20 minutes at 37° C. After staining, the cells were centrifuged at 400×g for 5 minutes and resuspended in 200 μl of autoMACS® running buffer, and evaluated using an SH800 cell sorter.

### 6.5.1.5. Lysosomal Staining

Lysosomes were stained with LysoTracker™ Red DND-99 (LysoTracker Red, L7528, Thermo Fisher Scientific, Inc.). Cells were suspended at a concentration of 3×10⁵ in 1 ml of culture medium and stained with 50 nM LysoTracker™ Red from the kit for 15 minutes at 37° C. After staining, the cells were centrifuged at 400×g for 5 minutes and resuspended in 200 μl of autoMACS running buffer, and evaluated using an SH800 cell sorter.

### 6.5.1.6. Lysosomal Proteolysis

Cells were incubated with 0.1 mg/ml DQ™ Green BSA (DQ BSA, D12050, Thermo Fisher Scientific, Inc.) for 4 hours, washed two times and incubated for 3 hours in fresh media to allow lysosomal accumulation of DQ™ BSA. Lysosomes were labeled with 50 nM Lysotracker™ Red for 15 minutes. After staining, the cells were centrifuged at 400×g for 5 minutes and resuspended in 200 μl of autoMACS® running buffer, and evaluated using an SH800 cell sorter.

### 6.5.1.7. Measurements of Respiratory Function and Glycolysis

An XFe96™ extracellular flux analyzer (Agilent Technologies, Santa Clara, CA, USA) was used to measure cellular respiratory function. Cells were suspended in Seahorse™ XF RPMI medium (Agilent Technologies) containing 10 mM glucose, 1 mM pyruvate, and 2 mM L-glutamine and seeded on XFe96™ 96-well microplates (101085-004, Agilent Technologies) coated with Cell-Tak™ (CLS354240, Corning Inc.) at a density of 1×10⁵ cells per well. After seeding, the cells were equilibrated in a non-CO₂ incubator for 20 minutes and used in the assay. For measurement of respiratory function, oligomycin (2 μM), carbonyl cyanide p-trifluoromethoxyphenyl hydrazone (FCCP, 2 μM) and rotenone/antimycin A (0.5 μM), which were adjusted using the reagents in the Seahorse™ XF Cell Mito Stress Test Kit (103015-100, Agilent Technologies), were sequentially added to each well after baseline measurement. The data are presented as the oxygen consumption rate (OCR; pmol/minute). Basal respiration, ATP production, maximal respiration, proton leakage, spare respiratory capacity, nonmitochondrial oxygen (non-MTC) and coupling efficiency were calculated using Wave Controller™ 2.4 (Agilent Technologies). For the measurement of glycolysis, glucose (10 mM), oligomycin (1 μM) and 2-deoxy-D-glucose (2-DG, 50 mM), which were adjusted using the reagents in the Seahorse™ XF cell glycolysis stress test kit (103020-100, Agilent Technologies), were sequentially added to each well after baseline measurement. The data are presented as the extracellular acidification rate (ECAR; mpH/minute). Glycolysis, glycolytic capacity and glycolytic reserve were calculated using Wave Controller™ 2.4.

### 6.5.1.8. Peritoneal Bacterial Quantification

The mice were euthanized with isoflurane at 24 hours post-CLP. The skin of the abdomen was cut open after disinfection, and without damage to the muscle layer, the peritoneal cavity was washed with 4 ml of sterile PBS with 2 mM EDTA. The obtained peritoneal lavage was diluted 1:10,000 in PBS, and 40μ of the diluted solution was plated on LB agar (22700-025, Thermo Fisher Scientific, Inc.) without any antibiotics. After incubation in a non-humidified incubator at 37° C. for 24 hours, the plates were photographed by a ChemiDoc Imaging System, and colony-forming units (CFUs) were counted in ImageJ™ (Version 1.53t, National Institutes of Health, Bethesda, MD, USA). The results are expressed as the number of CFUs per cm².

### 6.5.1.9. Subcellular Isolation for Western Blotting

THP-1 cells were resuspended in fractionation buffer (20 mM HEPES, 10 mM KCl, 2 mM MgCl2, 1 mM EDTA, 1 mM EGTA, 1 M DTT, 1/100 Protease Inhibitor Cocktail Set I (FUJIFILM Wako Pure Chemical Corporation), pH 7.2) and homogenized by passing twenty times through a 29-gauge needle. The lysate was maintained on ice for 20 minutes and then was separated into pellet containing nuclei and supernatant containing cytoplasm, membrane and mitochondria by centrifuging at 720 g for 5 minutes. The supernatant was centrifuged again at 12,000 g for 10 minutes. The cytoplasmic supernatant from the pellet was transferred to a clean tube. Nuclear pellet was washed with 500 μL of fractionation buffer and centrifuged again at 720 g for 10 minutes. The pellet in fractionation buffer was resuspended, then sonicated to shear genomic DNA and homogenize the lysate. These proteins were analyzed by Western blotting.

### 6.5.1.10. Western Blotting

Cytoplasmic protein was dissolved in RIPA buffer (182-02451, FUJIFILM Wako Pure Chemical Corporation), boiled for 10 minutes, electrophoresed through 10% Mini-PROTEAN® TGX Precast Protein Gels (4561036, Bio-Rad Laboratories Inc.) and electroblotted onto a PVDF transfer membrane (IPVH00010, Merck KGaA, Darmstadt, Germany). The membrane was blocked with PBS containing 5% skim milk and 0.05% Tween 20 (P1379, Merck KGaA) and incubated for 1 hour with a PGC1α (sc-517380, Santa Cruz Biotechnology, Dallas, TX, USA), TFEB (ab267351, Abcam plc., phospho-Akt (Ser473) (9271, Cell Signaling Technology, Inc.), PI13 kinase p85 (19H8) (4257, Cell Signaling Technology, Inc.), phospho PI13 kinase p85 (Tyr458)/p55 (Tyr199) (4228, Cell Signaling Technology, Inc.), S6 ribosomal protein (5G10) (2217, Cell Signaling Technology, Inc.), phospho-S6 ribosomal protein (Ser235/236) (2211, Cell Signaling Technology, Inc.), AMPKα (23A3) (2603, Cell Signaling Technology, Inc.), phospho-AMPKα (Thr172) (40H9) (2535, Cell Signaling Technology, Inc.), LC3 (0231-100BIOTIN/LC3-5F10, nanotools GmbH, Teningen, Germany), GAPDH (MAB374, Merck KGaA), and α-tubulin (66031-1-Ig, Proteintech Group, Inc., Rosemont). After washing, the membrane was incubated with a 1:5000 dilution of anti-mouse IgG (7076S, Cell Signaling Technology, Inc.) or anti-rabbit IgG HRP-linked antibody (7074S, Cell Signaling Technology, Inc.) in blocking buffer. Subsequently, the blots were developed using a Clarity™ Western ECL Substrate (1705060, Bio-Rad Laboratories Inc.) or Clarity™ Max Western ECL Substrate (1705062, Bio-Rad Laboratories Inc.), and the protein bands were visualized using a VersaDoc™ or ChemiDoc™ Imaging System (Bio-Rad Laboratories Inc.). Protein levels were quantified using ImageJ™.

### 6.5.1.11. Immunocytochemistry

Cells were fixed in 4% paraformaldehyde at 4° C. for 15 min. in the presence of a protein-blocking solution consisting of PBS supplemented with 5% normal goat serum (X090710-8, Agilent Technologies Inc., SantaClara, CA, USA). The cells were incubated overnight with anti-TFEB antibody (ab267351, Abcam pic.) in PBS at 4° C. The cells were washed extensively in PBS and incubated at room temperature for 30 min with an anti-rabbit IgG (H+L) antibody tagged with Alexa Fluor™ 488 (Thermo Fisher Scientific, Inc.). The nuclei were counterstained with 4′,6-diamidino-2-phenylindole (DAPI; diluted 1:500, #5748, FUJIFILM Wako Pure Chemical) in PBS at room temperature for 30 min. Fluorescence images were obtained using a Biorevo™ BZ-9000 fluorescence microscope (Keyence Corporation, Osaka, Japan). The identification of TFEB migrated to nuclear was performed using ImageJ™. First, the multicolor image was separated into TFEB- and DAPI-stained images. These images were converted to binarized images by thresholding, where a foreground pixel was assigned the maximum value of 255 and background pixels were assigned the minimum possible value of 80. The area where the TFEB and DAPI areas overlap is defined as the nuclear TFEB. The percentage of cells in the image with nuclear TFEB was calculated.

### 6.5.1.12. Mitochondrial Network Analysis (MINA)

Mitochondrial morphology analysis was performed using the Mitochondrial Network Analysis (MiNA) toolset, which was downloaded from github.com/stuartlab (Valente et al., 2017, Acta Histochem 119:315-326). To obtain precise results, the quality of the images was first improved. Choices for image preprocessing, including an unsharp mask and enhanced local contrast, are presented to the user through the MiNA interface. For analysis, the image was first binarized by thresholding, assigning a maximum value of 255 to foreground pixels and a minimum value of 0 to background pixels. Next, using Image™'s built-in skeletonization function, the binary image was converted into a skeleton that represents the features of the original image as a wireframe of one-pixel-wide lines. All pixels within a skeleton were then grouped into three categories: end point pixels, slab pixels, and junction pixels. The area of the mitochondrial footprint, slab pixels and junction pixels of individual cells was evaluated. The mitochondrial footprint is the number of pixels in the binary image containing signal multiplied by the area of a pixel if the calibration information is present.

### 6.5.1.13. Mitochondria Lysosome Contact Site Analysis

Analysis was performed using ImageJ™. First, the multicolor image was separated into images stained with MitoTracker™ Green and LysoTracker™ Red. These images were converted to binarized images by thresholding, where a foreground pixel was assigned the maximum value of 255 and background pixels were assigned the minimum possible value of 100. The contact site is the area where the regions of interest (ROIs) of the mitochondria and lysosomes overlap. The ratio of contact sites to the mitochondrial area of individual cells was evaluated.

### 6.5.1.14. Statistical Analysis

The results are presented as the means±standard deviations. The statistical significance of differences among groups was evaluated using parametric unpaired t tests for bar graphs. Mantel-Cox tests were used for statistical analysis of datasets of Kaplan-Meier survival curves (Prism™ 9 software, GraphPad Prism Software Inc., San Diego, CA, USA). P<0.05 was considered to indicate significance.

### 6.5.2. Results

### 6.5.2.1. Improved Survival and Anti-Inflammatory Effects of ZLN-005 in the CLP Sepsis Model

Cecal ligation perforation (CLP) has been shown to be the most appropriate animal model for polymicrobial sepsis and was used in this study. The severity of CLP can be controlled by the location of the ligation and the number of perforations. The ligation was performed at the base or middle of the cecum, and the severity was controlled by performing one or two perforations with a 20 Gauge needle beyond the ligature. A model in which the base of the cecum is ligated and two perforations are made has a 50% survival rate on postoperative Day 2 (data not shown). The effect of ZLN-005 on naïve THP-1 was observed at the mRNA and protein levels overtime, with a 2-fold increase in expression at 48 and 36 hours, respectively (data not shown). To examine the therapeutic effect of ZLN-005, the scheme was to inject ZLN-005 intraperitoneally for three consecutive days after CLP was performed in 10-20-week-old C57BL/6 mice (FIG. 1A).

In the sham surgery group, 25% of the mice died after 24 hours, while there were no deaths in the ZLN-005 group. At 72 hours, all sham-operated mice were dead; however, 40% of the ZLN-005-treated mice were alive, and 30% remained alive after 6 days, with Kaplan-Meier survival analysis results revealing significantly better survival in the ZLN-005 group (FIG. 1B). In addition, the behavior of the animals just 2 hours after the first drug administration after CLP was highly contrasting. In the sham-operated group, the mice remained in one place, were very unresponsive to stimuli and rarely moved. In the ZLN-005 group, however, mice spontaneously moved around and rarely stayed in one place, and they rapidly exhibited escape behaviors in response to stimuli. These early behavioral changes suggest an immediate effect of ZLN-005 on bacteria leaking into the peritoneal cavity in addition to its mechanism of action as a PGC1α activator at the transcriptional level. In the CLP model, the intraperitoneal macrophage-monocyte lineage has been reported to exhibit a decisive response in early pathogenesis, and the expression of Pgc1α, for which ZLN-005 has been reported, and Tfam, which plays an essential role in mitochondrial biogenesis, in intraperitoneal cells, including macrophages and monocytes, was examined. Both transcripts were significantly elevated (FIG. 1C).

In the CLP model, inflammatory cytokines were drastically increased from the early stage, and excess inflammatory cytokines played an important role in pathogenesis (FIG. 1D). Tnf-α and II1β were most highly expressed in the liver, followed by the kidney, lung, and heart in the sham group. 116 was most highly expressed in the kidney in the sham group but was significantly downregulated by ZLN-005 treatment, as were Tnf-α and II1β, to a level similar to that in the sham group. Although Infy expression itself was lower than that of other cytokines, the inhibitory effect of ZLN-005 was significant in the heart and lung, down to the expression level of the sham group. These results indicate a high anti-inflammatory effect of ZLN-005.

### 6.5.2.2. Organ Protection of ZLN-005 in the CLP Sepsis Model

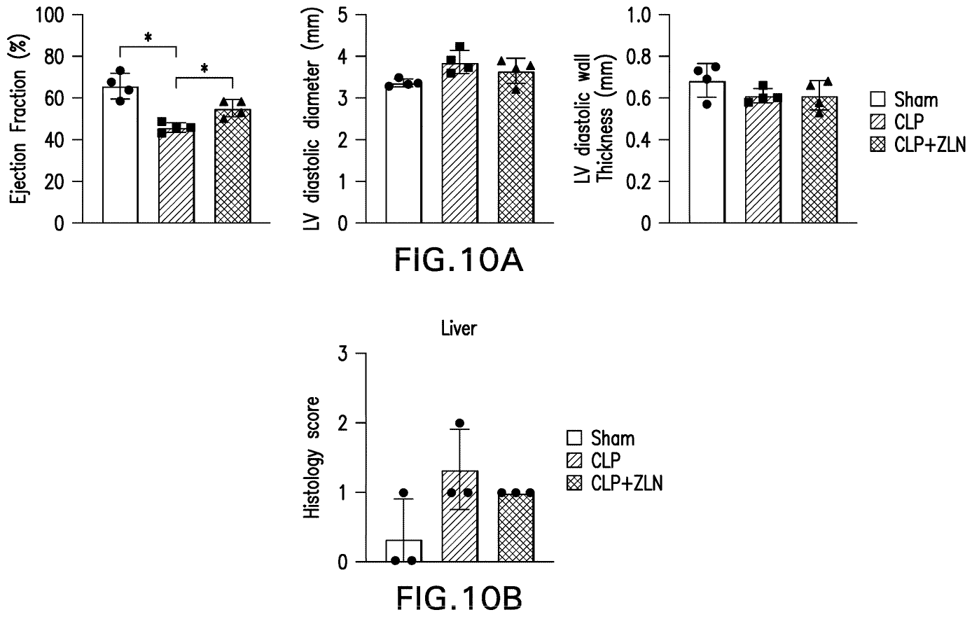

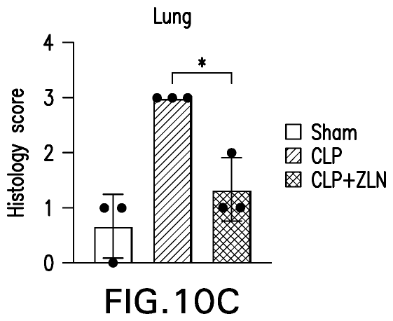

Twenty-four hours after CLP, ultrasound cardiography was performed. The sham group showed an ejection fraction from the 60% to 40% range, while in the group treated with ZLN-005, the ejection fraction remained in the 50% range (FIG. 10A). Cardiac pathological examination revealed no significant findings, including cellular infiltration or hemorrhage, suggesting that dysfunction could be caused by fluid factors such as proinflammatory cytokines and coagulation factors and that the suppression of cytokine storms by ZLN-005 could work to preserve cardiac function (data not shown). The kidneys, like the heart, showed no significant changes (data not shown). The liver, however, showed a disorganized lobular structure, hemorrhagic lesions, and cellular infiltration in the sham group and slightly improved findings in the group treated with ZLN-005 (data not shown). Quantification failed to reveal statistically significant changes (FIG. 10B). The spleen showed a large disruption of follicular structure in the sham group, while the follicular structure was well preserved in the group treated with ZLN-005 (data not shown). The lungs showed the most pronounced pathology, with cellular infiltration clearly suppressed in the ZLN-005 group, the interstitium preserved, and edema very mild (data not shown). Quantification also showed significantly less damage in the ZLN-005 group (FIG. 10C).

The effect of ZLN-005 on the elimination of intraperitoneal bacteria in the CLP model was also investigated. Ascites was collected 2 and 24 hours after CLP treatment, and colonies were measured by bacterial culture. At 2 hours, the number of colonies had already decreased by more than half in the ZLN-005-treated group, and by 24 hours, the number of colonies had further decreased by about one-fourth (data not shown). This indicates that ZLN-005 promotes the elimination of bacteria released into the peritoneal cavity. These results suggest that ZLN-005 was able to make the intraperitoneal monocyte-macrophage lineage more controllable against an uncontrollable bacterial load. The early onset of action at 2 hours suggests that the mechanism of action is directly related to metabolism and digestion rather than requiring a process of transcription and translation.

### 6.5.2.3. Mitochondrial Functional Alterations by ZLN-005

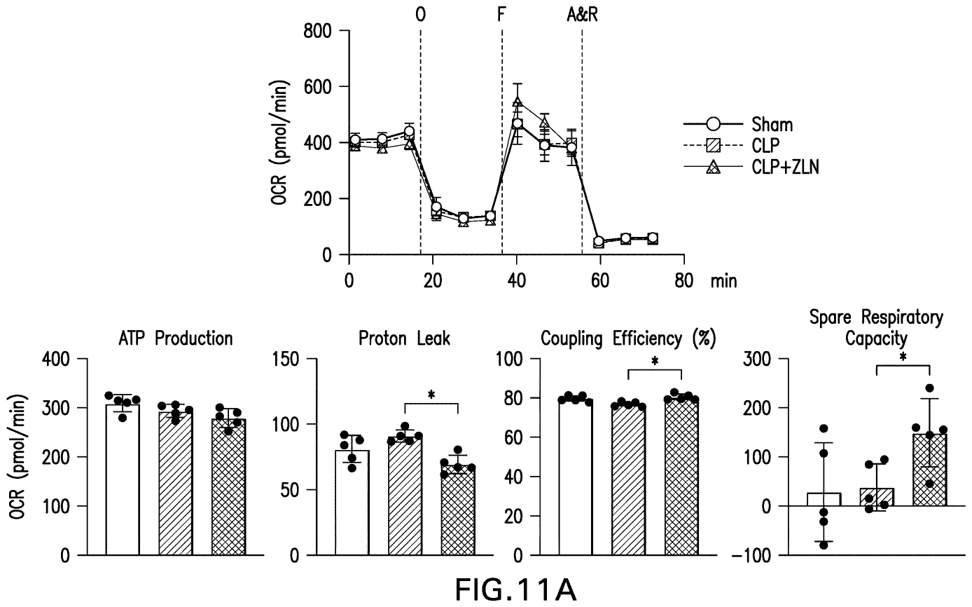

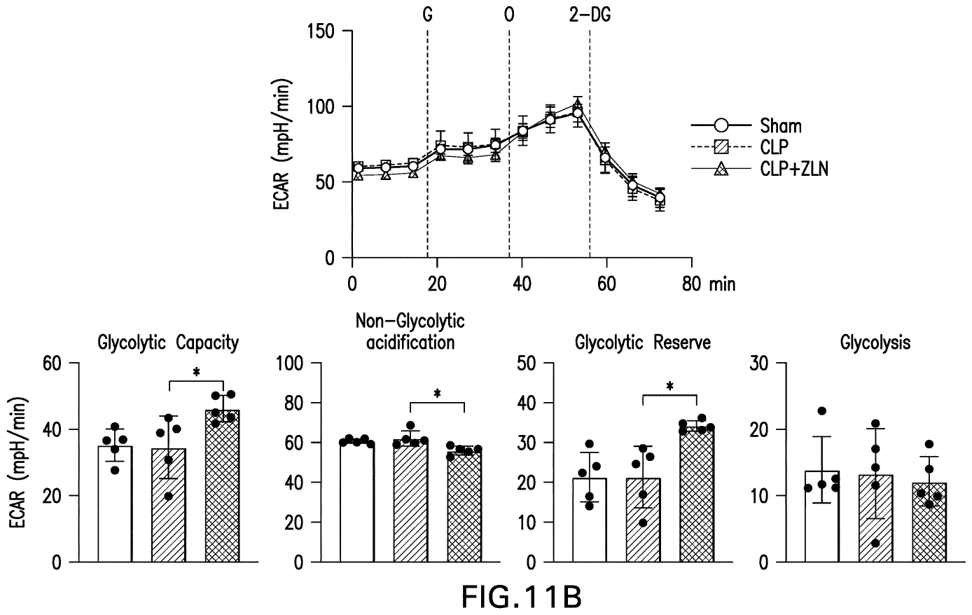

In the CLP model, the mitochondrial respiratory capacity and metabolic capacity of the glycolytic system in intraperitoneal cells after i day of treatment were examined. Intraperitoneal cells after CLP treatment were harvested and profiles of mitochondrial oxidative phosphorylation (OXPHOS) and glycolytic capacity were obtained by sequential use of respiratory chain complex inhibitors and glycolytic system inhibitors using Seahorse™. Intraperitoneal cells on postoperative day 1 did not show significant changes in OXPHOS with CLP, but administration of ZLN-005 decreased proton leak, resulting in a significant increase in coupling efficiency. In addition, spare respiration capacity was significantly increased (FIG. 11A). On the other hand, in the glycolytic system, as in OXPHOS, CLP treatment did not cause significant changes on the first postoperative day, but administration of ZLN-005 significantly increased glycolytic capacity and glycolytic reserves (FIG. 11B). This indicates that ZLN-005 is responsible for the improvement in reserve capacity of both metabolic pathways.

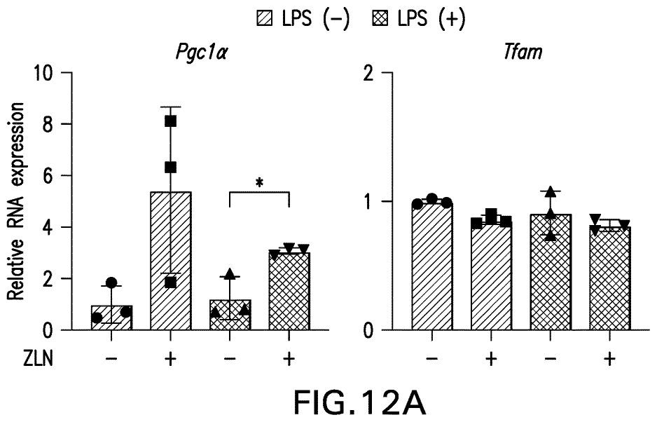

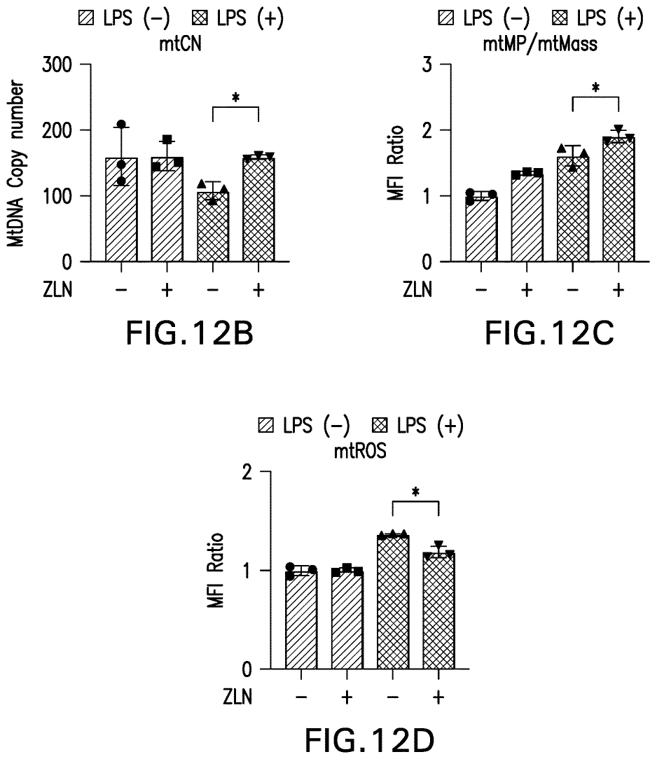

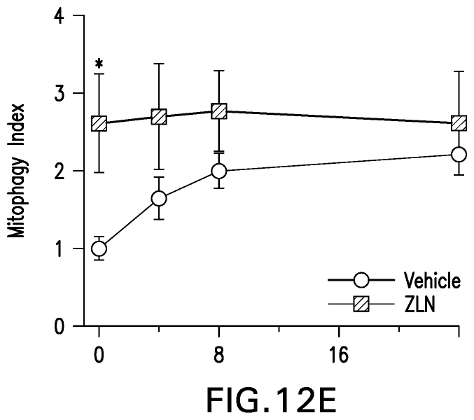

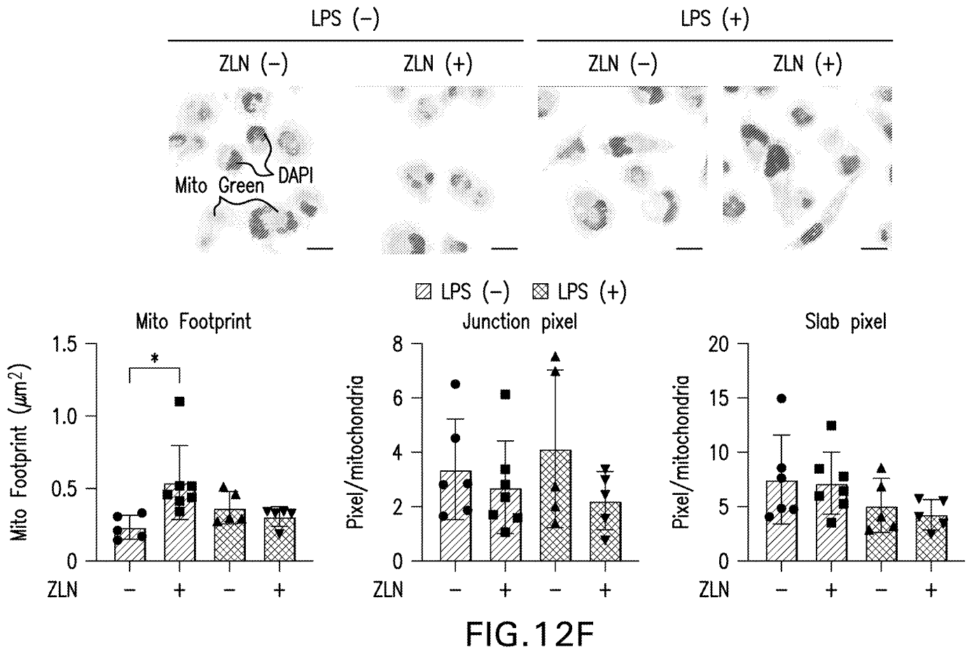

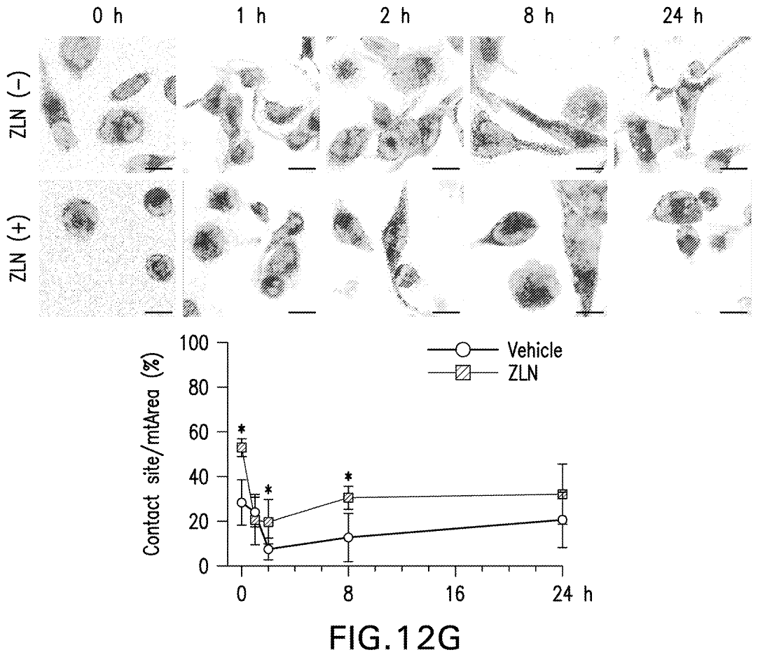

The mechanism of action of ZLN-005 proposed to date has been to enhance PGC1α at the transcriptional level, but there were no significant changes in the OXPHOS profile other than reserve capacity, and no alterations leading to a dramatic improvement in sterilization of the abdominal cavity. Therefore, the mitochondrial function of ZLN-005 was examined using THP-1, a human macrophage strain, in order to dissect alternative mode of action of ZLN-005 in this model. The effect of ZLN-005 on THP-1 was examined in the model of stimulation with LPS at 24 hours after stimulation. Pgc1a mRNA was significantly elevated at the transcriptional level with or without LPS stimulation by ZLN-005, whereas Tfam mRNA showed no significant change in either group (FIG. 12A). However, mtDNA copy number was decreased by LPS stimulation but was significantly restored to that of the untreated group by treatment with ZLN-005 (FIG. 12B). PGC1α is a major regulator of mitochondrial biogenesis, and mitochondrial DNA copy number as a phenotype was examined. A significant increase in copy number was observed with ZLN-005 (FIG. 12B). Mitochondrial mass was measured by MitoGreen and showed no significant changes with LPS stimulation or ZLN-005 administration (data not shown). Mitochondrial membrane potential (mtMP) by TMRM (data not shown) was corrected by mitochondrial mass, and mtMP/mtMass was increased by LPS stimulation and further increased by ZLN-005 administration (FIG. 12C). Mitochondrial ROS was significantly increased by LPS stimulation, but the increase was reversed by ZLN-005 (FIG. 12D). To measure mitophagy, THP-1 cells were generated which constantly express MitoKeima Red, and the effect of ZLN-005 under LPS stimulation overtime was examined. A slight induction of mitophagy was detected as early as 2 hours after ZLN-005 administration, and no increase in mitophagy was recognized overtime (FIG. 12E). This enhanced mtDNA replication and increased mitophagy suggest enhanced mitochondrial turnover, but only to a small extent under the conditions of this study. Without being bound by theory, mitochondrial turnover might not contribute significantly to cellular phenotypic changes.

Because ZLN-005 significantly affects mitochondrial biogenesis and turnover under LPS-stimulated conditions, its effect on mitochondrial dynamics using THP-1 cells was examined. Mitochondria were stained with TMRM to quantify mitochondrial morphology using ImageJ™. Footprints indicating mitochondrial mass were more sensitive than those from MFI of MitoGreen™ by FACS (data not shown), and ZLN-005 was slightly increased in the unstimulated state but showed no change in the LPS-stimulated state (FIG. 12F). The four groups showed no change in Slub pixel and Junction pixel, which are indicators of branching status (FIG. 12F). The proximity of the mitochondria and lysosomes was assessed with dye co-staining using dyes that stain the two organelles. The cross-section with the largest lysosomal staining area was selected and the co-staining area at that cross-section was calculated. Although only one cross-section was evaluated, a clear increase in co-staining area was observed with exposure to ZLN-005 throughout 24 hours following LPS exposure. Without being bound by theory, these findings suggest tethering of the two organelles and suggest that lysosomes may be involved in another mechanism of action of ZLN-005 (FIG. 12G).

### 6.5.2.4. Lysosomal Alterations by ZLN-005

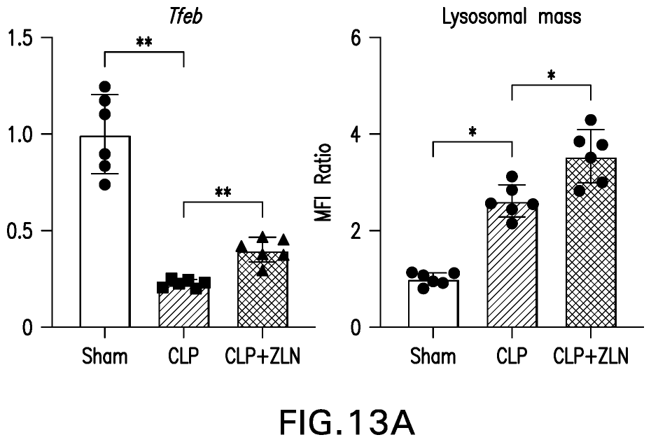

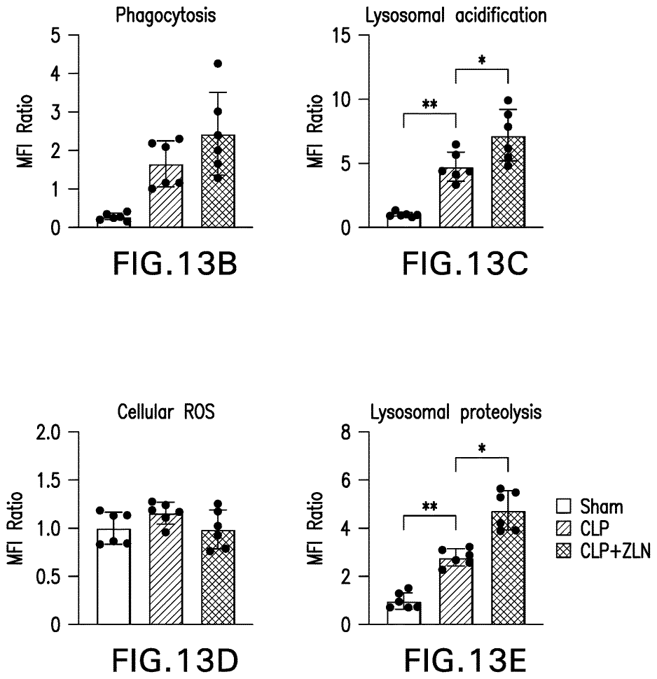

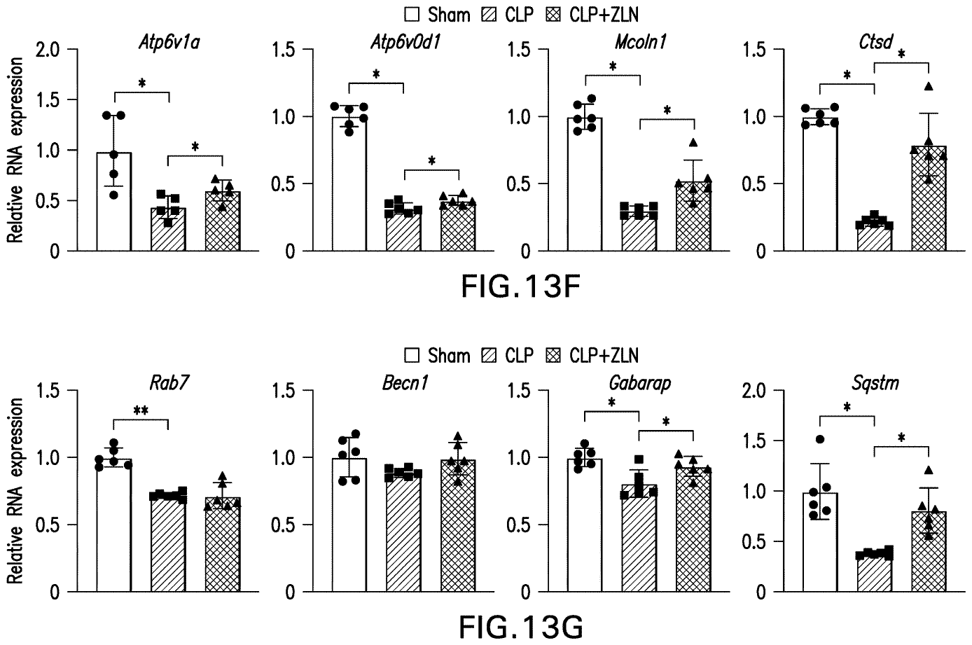

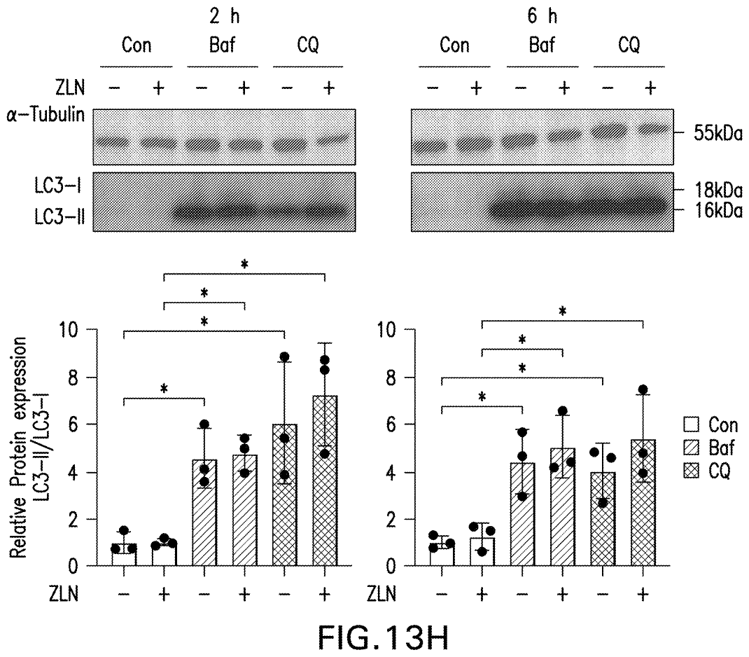

Subsequent studies were conducted to evaluate the hypothesis that phagocytic cells may have enhanced the process from phagocytosis to digestion through alteration of the phenotype by responding rapidly to an enormous bacterial load. Intraperitoneal cells were harvested from the CLP model and used as material for studies ranging from phagocytosis to lysosomes. Although the lysosome contains hydrolases that digest pathogens and macromolecules, it is necessary to expand the volume of the phagolysosome and maintain its lumen at approximately 4.6 pH, the optimum for these hydrolases, to process large amounts of pathogens. First, Tfeb mRNA expression was examined, which was markedly decreased by CLP alone and significantly increased by treatment with ZLN-005 but was still lower than that in the sham group (FIG. 13A). Lysosomal mass was increased by CLP alone and further enhanced by treatment with ZLN-005 (FIG. 13A). Intraperitoneal cells were incubated with fluorescent dextran, and the fluorescence intensity was evaluated as the degree of phagocytosis. Phagocytosis was significantly elevated in the CLP group, and ZLN-005 showed further elevation, although no significant difference was recognized (FIG. 13B). Next, the final stage of bacterial killing and the intraluminal alterations of the lysosome was examined. Lysosome pH is one of the most fundamental requirements for lysosome function, and the enhancement of acidity was measured as an increase in fluorescence intensity using a pHrod. Lysosomal acidity was enhanced in the CLP group and was further significantly enhanced by treatment with ZLN-005 (FIG. 13C). However, the expression of intracellular ROS, as measured by CellRox™, was not significantly elevated during this period (FIG. 13D). The acidic shift of the lysosome pH was linked to the promotion of the function of hydrolases, as shown by the use of DQ™-BSA, in which the fluorescent dye is released from quenching inhibition and excites fluorescence when proteolysis is enhanced. Although a certain degree of proteolysis occurs in unstimulated cells by the addition of DQ™-BSA, the degree of proteolysis is enhanced by LPS exposure, and is further enhanced by the administration of ZLN-005, as observed by fluorescence microscopy (FIG. 13E), and quantification by FACS showed that ZLN-005 promoted proteolysis with significant differences (FIG. 13E). Without being bound by theory, these results suggest that in the present model, the oxidative burst is not significantly involved in pathogen eradication after i day of infection, but rather the hydrolytic processing is considered to be a major contributor.

The mRNAs of most lysosomal proteins that possess the CLEAR motif in the promoter, which is a target of TFEB, were elevated compared with those in the CLP group. In particular, all mRNAs of hydrolases, such as Ctsd, and membrane proteins, such as Atp6v1A, Atp6v0d1, and Mcoln1, were significantly elevated (FIG. 13F). With respect to autophagy, Becni1 and Gabarap were only slightly elevated, and Rab7 was not changed by the administration of ZLN-005, although a significant increase was recognized on Sqstm/p62 (FIG. 13G). Without being bound by theory, the analyses of these transcripts suggest that ZLN-005 could be more involved in phagolysosome acidification than in xenophagy. Bacteria killing in phagocytes can be achieved by fusion of phagosomes with lysosomes or by xenophagy, in which the host triggers autophagy when the phagosomes are damaged by the escape behavior of the bacterium. In the LPS model of THP-1, autophagy flux in the presence of bafilomycin A1, an ATPase inhibitor, and chroloquine, a fusion inhibitor of autophagosomes and lysosomes, using LC3 conversion as an indicator was examined. The autophagy flux was measured after 6 hours of exposure to LPS followed by 2 or 6 hours of treatment with the autophagy inhibitor. Neither LC3-II nor LC3-II/I ratios were increased by treatment with ZLN-005 (FIG. 13H). The effects of the two inhibitors were common in this system, with LC3-II and LC3-II/I ratios increasing with significant differences, while ZLN-005 had no significant effect on the increase of the two inhibitors to LC3-II and LC3-II/I ratios. Without being bound by theory, these results indicate that the effect of ZLN-005 on macroautophagy is not significant.

### 6.5.2.5. Regulation of TFEB

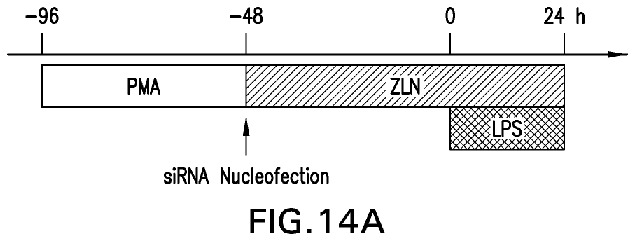

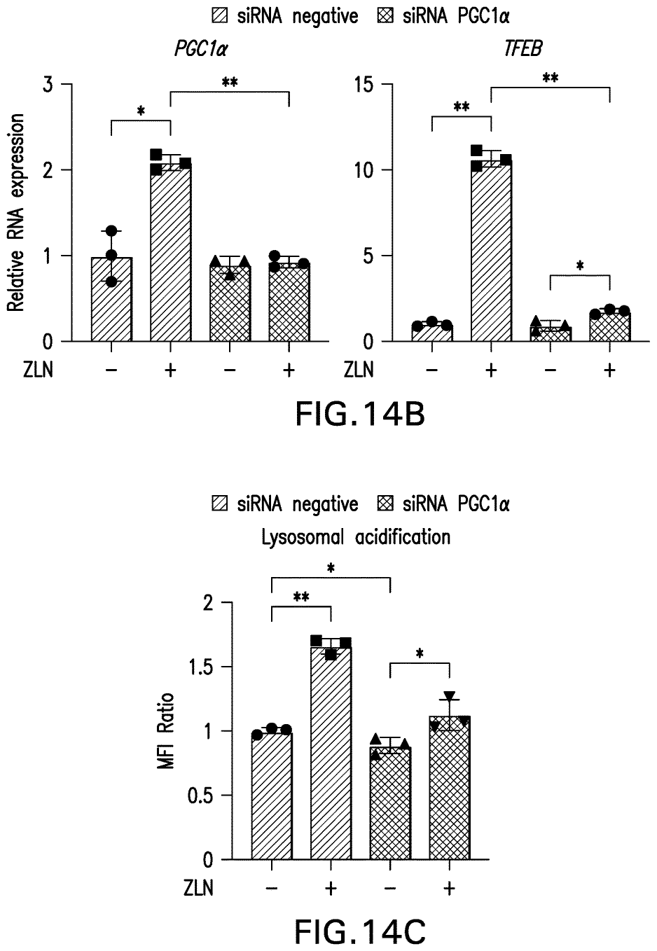

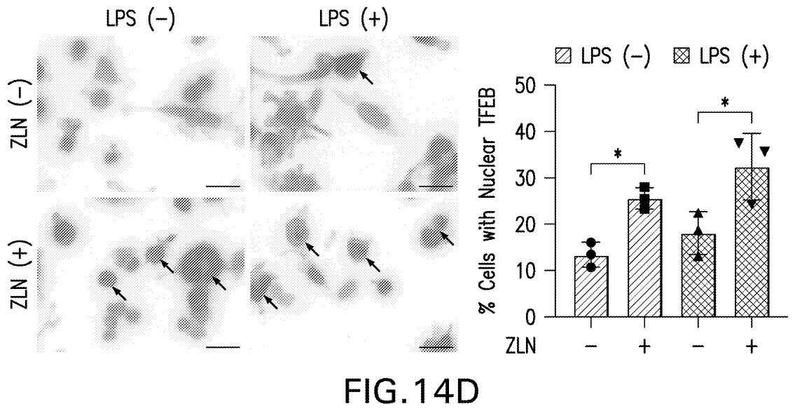

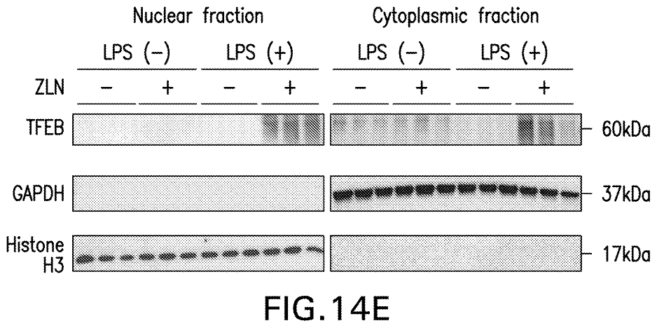

PGC1α knockdown (KD) did not significantly change the mRNA level of Tfeb in THP-1 with LPS stimulation alone (FIG. 14B). When ZLN-005 was added to LPS stimulation, Tfeb mRNA levels were markedly increased and PGC1α KD caused an increase in Tfeb mRNA levels, but the rate of increase was greatly reduced (FIG. 14B). Without being bound by theory, these results suggest that PGC1α plays a role in the regulation of TFEB at the transcriptional level upon treatment with ZLN-005. Lysosomal acidification was not altered by PGC1α KD when stimulated with LPS alone, and was significantly elevated when ZLN-005 was administered, although the increase in acidity was slightly reduced (FIG. 14C). Without being bound by theory, this suggests that ZLN-005 may have a mechanism of action other than transcriptional regulation of Tfeb.

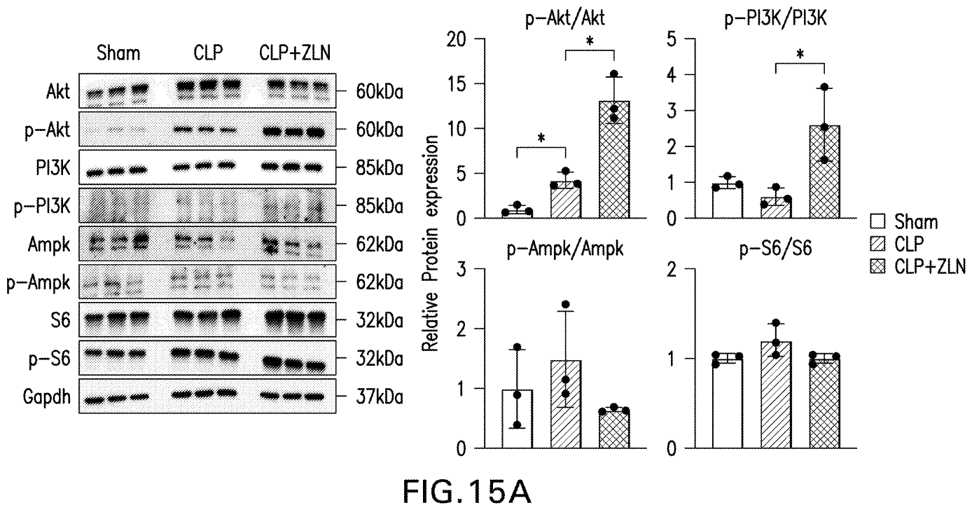

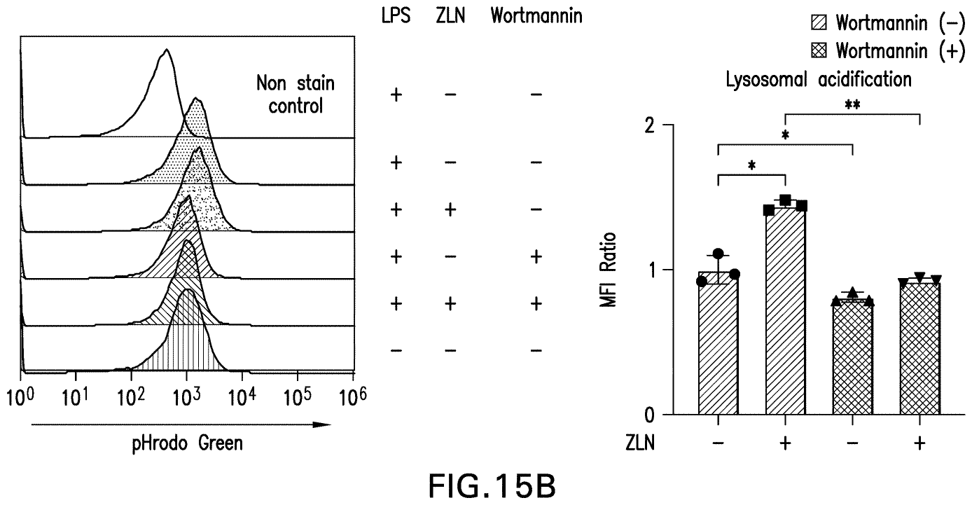

Alternatively, Tfeb is always present in the cytoplasm, and its nuclear translocation is regulated by phosphorylation, where it acts as a transcription factor. It was considered if there might be intervention of ZLN-005 in this pathway and performed fluorescent immunostaining of TFEB in THP-1 by LPS exposure and ZNL-005 administration (FIG. 14D). Quantification showed that nuclear migration proceeds in the absence of LPS exposure by ZLN-005, but is further enhanced by LPS exposure with ZLN-005 (FIG. 14D). To further confirm the nuclear migration of TFEB, cells were collected and divided into nuclear and cytoplasmic fractions and Western Blotting was performed to examine the presence of TFEB; in the presence of LPS exposure, nuclear migration of ZLN-005-induced TFEB was quite pronounced (FIG. 14E). Finally, the molecular pathway of action in ZLN-005 was examined and found that among the various factors that promote V-ATPase V0-V1 assembly, the PI13K/AKT axis is the pathway most closely involved in phagocytosis. The involvement of mTORC1, which is downstream of this pathway, and AMPK, which is closely linked to the regulation of both PGC1α and TFEB, was also investigated using WB, demonstrating that PI3K and AKT were markedly phosphorylated and activated by ZLN-005 treatment (FIG. 15A). However, no activation of S6 downstream of mTORC1 was observed, and the activity of AMPK was hardly changed by ZLN-005 administration (FIG. 15A). Both mTORC1 and AMPK are deeply involved in autophagy regulation, and ZLN-005 did not activate either of them in this study. Since the PI3K-AKT pathway is greatly activated by ZLN-005, which may play a major role in the phosphorylation of TFEB, it was evaluated whether the PI3K inhibitor Wortmannin could cancel the change in lysosomal acidification. ZLN-005 did cause acidification of lysosomes in the absence of Wortmannin, but did not cause acidification in the presence of Wortmannin (FIG. 15B). Without being bound by theory, these results suggest that ZLN-005 causes lysosomal acidification through PI3K, which could promote V0-V1 assembly.

### 6.5.2.6. Mode of Action of ZLN-005

Without being bound by theory, it is believed that the mode of action of ZLN-005 in bacterial infections is as follows:

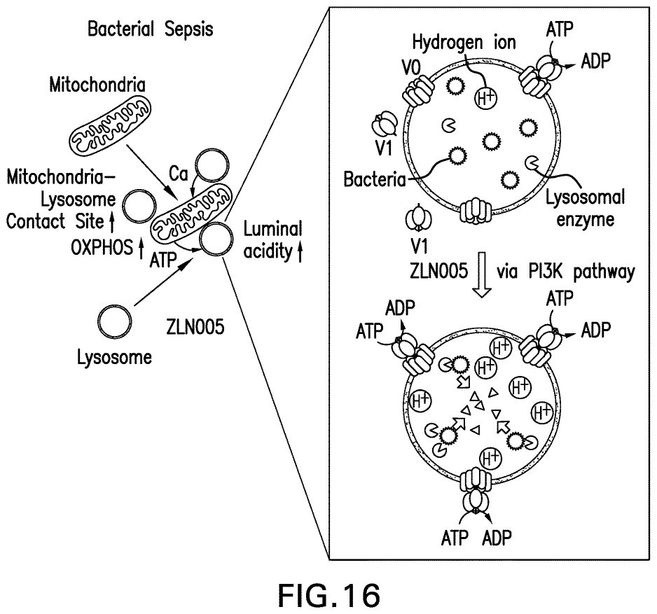

ZLN-005 promotes the decay of incorporated bacteria by acidifying the lysosomal pH in macrophages and by promoting lysosomal biogenesis (FIG. 16). With regard to the former, V-ATPase, which consists of the V0 complex present in the lysosomal membrane and the V1 complex present either in the cytosol or bound to the V0 complex, V0-V1 assembly is promoted by the presence of ZLN-005. To drive the V-ATPase, the mitochondrial biogenesis-promoting action of ZLN-005 increases ATP production. The two organelles are also brought into close proximity by the action of PI3K. On the other hand, in the latter case, PI3K induces the transformation of phosphatidylinositides, and TFEB is dephosphorylated and transferred into the nucleus using Ca as a second signal. This promotes lysosomal biogenesis and the production of different hydrolases present in the membrane proteins of the lysosome and in the lumen. All of these events aid in the complete digestion of the incorporated bacterium.

### 6.5.3. Discussion

This Example confirms that intraperitoneal administration of ZLN-005 is therapeutic for polybacterial sepsis and shows, without being bound by theory, that the molecular mechanism of the effect is the enhancement of lysosomal acidity and biogenesis through TFEB. Again without being bound by theory, the upstream signal for TFEB activation could be PI3K. This Example indicates that ZLN-005 has a dual action on two intracellular organelles, mitochondria and lysosomes, and their mechanism of action could be coordinated. This Example further indicates that for bacterial clearance in the early phase of sepsis, ZLN-005 has a mechanism of action that contributes to the improvement of the disease state.

In this Example, the induction of Tfeb mRNA by ZLN-005 under LPS stimulation was suppressed by PGC1α KD, indicating that Tfeb mRNA is regulated by PGC1α. On the other hand, the enhancement of TFEB migration to the nucleus by ZLN-005 treatment was observed by immunofluorescence staining and western blotting analyses in the nuclear and cytoplasmic fractions, suggesting that ZLN-005 acts directly on the nuclear migration of TFEB. The action of ZLN-005 on lysosomes via TFEB, in addition to the reciprocal enhancement of PGC1α at the transcriptional level, may be a mechanism to promote nuclear translocation of TFEB that is sustained for a period of time from very early pathological changes.

V-ATPase, the molecule responsible for lysosomal acidification, has been shown to be regulated by V0-V1 reversible disassembly, but its role in pathogenesis has been largely focused on cancer. It is a significant impact that this Example sheds light on the role of V-ATPase in sepsis. In sepsis, the inability of the lysosomal system to adequately respond to excessive bacterial load could be a factor in early mortality, and the dysfunction of V0-V1 ATPase assembly might be the molecular mechanism responsible for this inability. In this Example, ZLN-005 promoted bacterial killing to enhance survival with strengthening lysosomal acidity, suggesting that one of the molecular bases of innate immunity to sepsis is the V0-V1 assembly.

Without being bound by theory, the pathway by which ZLN-005 improved survival in the CLP model appears to be by promoting bacteria killing in the phagolysosomes. Regarding what directly executes the process in the final stages of bacterial killing, lysosome acidity, luminal hydrolases, and ROS are considered. The lack of a significant effect of ZLN-005 on cellular ROS and the significant increase and activation of hydrolases observed in this Example suggest that the bacterial clearance could be attributed to hydrolases, rather than cellular ROS.

In this Example, ZLN-005 strongly induced TFEB, a master gene that stimulates the production of membrane proteins and hydrolytic enzymes in the lysosome, which in turn act as an increase in lysosomal mass. TFEB is a transcriptional regulator with a positive feedback loop that rapidly responds to environmental changes mainly by posttranslational modification and transcriptionally enhances PGC1α. The stability and translocation of TFEB from the cytoplasm to the nucleus is regulated by phosphorylation by various kinases and dephosphorylation by phosphatases, and it translocates to the nucleus to promote the expression of its own genes and genes involved in autophagy and lysosome biogenesis. Many signal regulators negatively regulate TFEB, including mTORC1, but calcineurin positively regulates TFEB. PIKFYVE on the lysosome is a kinase that receives signals from AKT, which substrates PI3P to generate PI(3,5)P2. It has been reported that PI(3,5)P2 activates TRPML1, which is encoded by MCOLN1, and TRPML1 releases Ca2+ from lysosomes into the cytoplasm. Its increased concentration activates calcineurin, resulting in the dephosphorylation of TFEB to act as a transcription factor. The involvement of PI3K in the mechanism of action of ZLN-005 was indirectly demonstrated by enhanced phosphorylation of PI3K and AKT in the CLP model. In addition, lysosomal acidification by ZLN-005 was inhibited by the PI3K inhibitor wortmannin, providing direct evidence that ZLN-005 acts through PI3K. These factors, including PIKFYVE, TRPML1, and calcineurin, may be involved and contribute to the activation of TFEB by ZLN-005.

Signals from PI3K are transduced via AKT to mTORC1, which leads cells to anabolism, including proliferation, through phosphorylation of various factors. However, mTor signals suppress all autophagy processes, not only initiation and nucleation but also autophagosome elongation, maturation, and termination. Some bacteria have acquired the art of escaping innate immunity by exploiting this mTORC1 suppression of the autophagy process. Metabolism in sepsis results in a surge in energy demand, which activates AMPK, and this signal is inhibitory for mTORC1 via Rheb through phosphorylation of TSC2. Furthermore, AMPK supports autophagy through phosphorylation of ULK1 independent of mTOR. In this Example, the S6 phosphorylation pathway downstream of mTORC1 was not implicated in sepsis pathogenesis, and further activation of PI3K by ZLN-005 did not significantly after the signal downstream of mTORC1. However, the phosphorylation of AMPK, which acts antagonistically with mTORC1, is activated in the sepsis condition but did not change in this Example. This is in accordance with the fact that autophagy-related gene expression was only slightly altered in the early phase of sepsis in this Example.

It was shown in this Example that ZLN-005 causes an increase in mitochondrial lysosome contact sites and that OXPHOS is enhanced. Physical contact between these two intracellular organelles has long been reported to occur in a mitophagy-like degradative process, but it has recently been recognized that transient contact exists physiologically as a nondegradative process. The present Example shows that LPS-stimulated THP-1 by ZLN-005 causes tethering of two organelles from as early as 1 hr to as late as 24 hr. Although the molecular mechanism by which ZLN-005 promotes the coordination of the two organelles remains to be elucidated, the enhancement of physical contact might allow (without being bound by theory) the dual function of mitochondria and lysosomes to work more organically.

For sepsis, much effort is focused on controlling the hyperimmune reaction. However, drug development to improve the efficiency of bacterial clearance in the early phase has been stagnant. This Example shows that ZLN-005 improved overall survival by improving the pathophysiology of the early phase of the disease, addressing a long-felt need in the art. Furthermore, the dual action of ZLN-005 on mitochondria and lysosomes shown in the Example supports the use of ZLN-005 for treating lysosomal acidification failure-based neurodegenerative diseases, including Alzheimer's disease, Parkinson's disease, renal tubular acidosis, diabetes mellitus, Zimmermann-Laband syndrome (a condition associated with V1B2 mutation), and Cutis laxa type II and wrinkly skin syndrome. Without being bound by theory, it is believed that diseases that converge on lysosome acidification dysfunction as the molecular basis, albeit in a different pathology than sepsis, are therapeutic targets for ZLN-005 and related compounds.

### 6.6. Example 6: ZLN-005 Reduces Bacterial Increase in CLP-Induced Sepsis Model

This Example provide further evaluation of ZLN-005 and its ability to increase bacterial killing in a mouse cecal ligation and perforation (CLP) model.

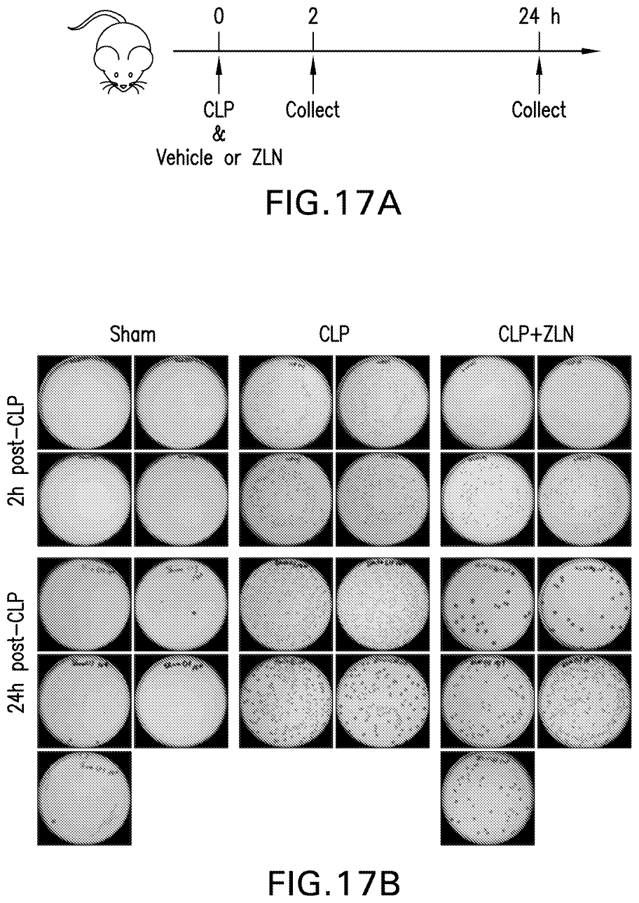

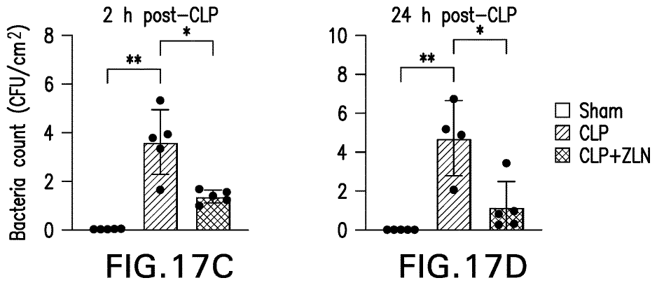

CLP was performed on mice at study hour 0 as described in Section 6.5.1.1. Mice were administered either ZLN-005 at 12 mg/kg (i.p.) or vehicle (DMSO) at the time of cecal ligation and perforation. Peritoneal ascites from each mouse were collected at study hours 2 and 24 and assayed for peritoneal bacteria levels (FIG. 17A).

Results are shown in FIGS. 17B-17D. Ascites from vehicle-treated CLP mice displayed a marked increase in bacteria count at 2 and 24 hours relative to the ascites from the sham-treated mice. Ascites from ZLN-005-treated CLP mice showed a reduction in the bacterial increase at both time points compared to vehicle-treated CLP mice.

### 6.7. Example 7: ZLN-005 Increases v-ATPase Assembly

Vacuolar-ATPases (v-ATPases) are ATP-driven proton pumps that function to acidify intracellular compartments and transport protons across the plasma membrane. In lysosomes, v-ATPases function to increase acidity in the lysosomal lumen. The activity of V-ATPase has been shown to be regulated by V0-V1 reversible disassembly.

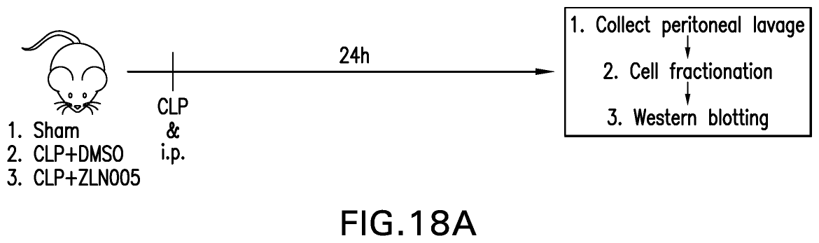

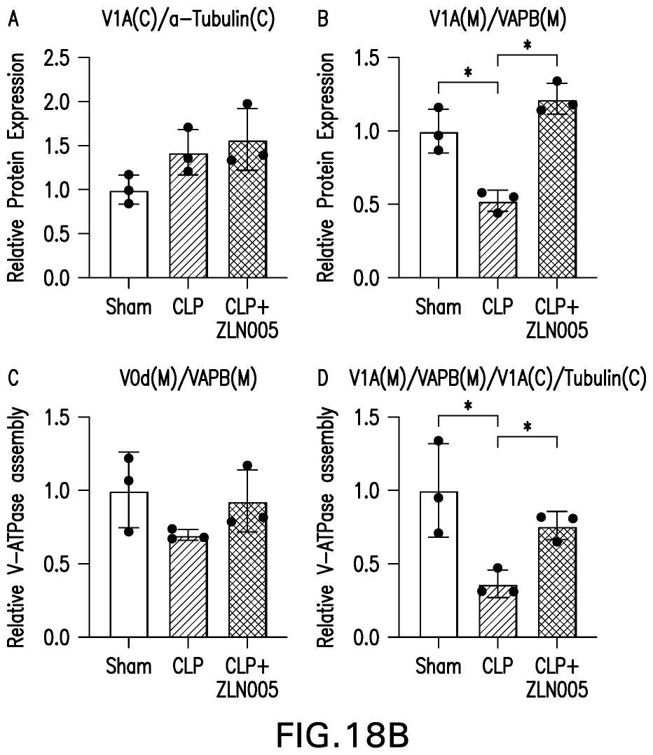

To assess the effect of ZLN-005 on v-ATPase assembly, CLP was performed on mice at study hour 0 as described in Section 6.5.1.1. Mice were administered either ZLN-005 at 12 mg/kg (i.p.) or vehicle (DMSO) at the time of cecal ligation and perforation. Peritoneal cells were collected at 24 hours. After cell fractionation, western blot was conducted as described in Section 6.5.1.10. to determine levels of V0 and V1 on the lysosomal membrane (FIG. 18A).

Assembly of v-ATPase involves association of V0 and V1 subunits, wherein V0 is the membrane-bound subunit to which V1 binds to form a v-ATPase. Without being bound by theory, the level of V1 protein associated with lysosome membrane can be indicative of the amount of fully assembled v-ATPases on lysosomes. V1 protein level in the cytosol was assessed by normalizing the expression of cytoplasmic V1A protein by cytoplasmic tubulin expression, which did not significantly differ between sham-treatment and CLP+vehicle or CLP+ZLN-005 (FIG. 18B). Expression of V1A protein relative to VAPB, indicative of V1 levels associated with lysosomes, was significantly lower in the CLP+vehicle samples than samples collected from sham-treated mice, suggesting a decrease in the number of fully assembled v-ATPases on lysosomal membranes. This decrease was reversed in samples from CLP mice treated with ZLN-005 (FIG. 18B). There was no significant difference in membrane bound V0 proteins in the treatment groups (FIG. 18B). Finally, the V0/V1 assembly was assessed by normalizing the lysosome-associated V1 by cytosolic V1, the results of which showed that CLP significantly decreased the association of V0 and V1, whereas ZLN-005 treatment diminished this decrease (FIG. 18B).

### 6.8. Example 8: ZLN-005-Mediated Lysosomal Acidification Under LPS Stimulation is Abolished by TRPML1 Inhibition

As described in Example 5, nuclear levels of TFEB regulate lysosomal biogenesis and autophagy. TRPML1 is a protein that activates cytosolic Ca²⁺ release leading to TFEB nuclear translocation.

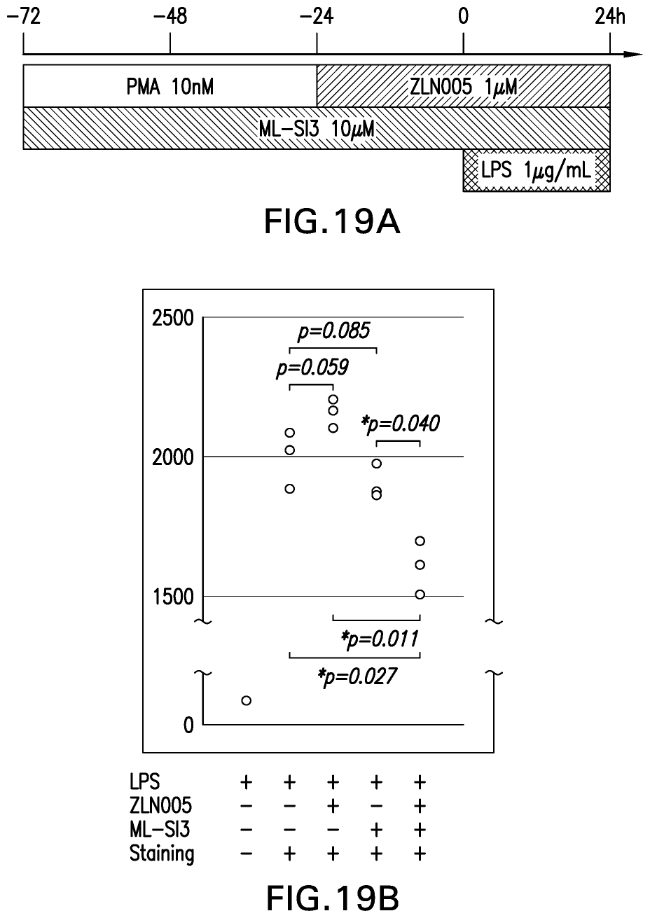

The role of TRPML-1 on ZLN-005-associated increase in lysosomal acidification was assessed in THP-1 cells. After macrophage differentiation, the cells were treated with the indicated concentrations PMA, ZLN-005, the TRPML1 inhibitor ML-S13, and LPS for the durations shown in FIG. 19A. Cells were sorted by FACS, wherein pHrod fluorescence was used as an indicator of lysosomal pH.

Results are shown in FIG. 19B. Level of pHrod fluorescence was higher in ZLN-005 and LPS treated cells relative to LPS-only cells. ML-S13 and LPS treated cells had pHrod fluorescence levels comparable to those in LPS-only cells. LPS-stimulated cells that were treated with both ZLN-005 and ML-S13 displayed a marked decrease in pHrod fluorescence.

### 7. SPECIFIC EMBODIMENTS

The present disclosure is exemplified by the specific embodiments below.

1. A method of treating a subject having a disease or disorder associated with impaired lysosomal acidification, comprising administering to the subject a therapeutically effective amount of an agent which is a compound of Formula (I):

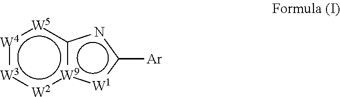

or a salt, hydrate, deuterated analog, or fluorinated analog thereof, wherein:
Ar is

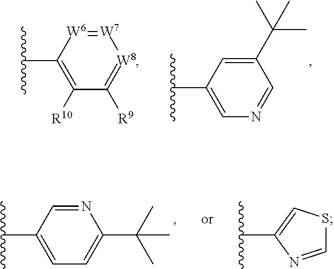

W¹ is N—R¹, O, or S, or when W⁹ is N, W¹ may additionally be C—R⁵⁰;
W² is C—R² or N;
W³ is C—R³ or N;
W⁴ is C—R⁴ or N;
W⁵ is C—R⁵ or N;
W⁶ is C—Re or N;
W⁷ is C—R⁷ or N;
W⁸ is C—R⁸ or N;
W⁹ is C, or when W¹ is C—R⁵⁰, W⁹ may be N;
R¹ is H, (C₁-C₃)alkyl, CH₂OC(═O)R³⁰, CH₂OP(═O)OR⁴⁰OR⁴¹, C(═O)OR⁴², or C(═O)R⁴³;
each of R², R³, R⁴, and R⁵ is independently hydrogen, deuterium, halogen, perfluoro(C₁-C₄)alkyl, (C₁-C₄)alkyl, (C₁-C₄)alkoxy, perfluoro(C₁-C₄)alkoxy, (C₁-C₄)acyl, (C₁₋₄)alkoxy(C₁-C₄)alkyl, hydroxy(C₁-C₄)alkyl, hydroxy, carboxy, (C₁-C₄)alkoxycarbonylamino, carboxamido, (C₁-C₄)alkylaminocarbonyl, cyano, acetoxy, nitro, amino, (C₁-C₄)alkylamino, di(C₁-C₄)alkylamino, mercapto, (C₁-C₄)alkylthio, aminosulfonyl, (C₁-C₄)alkylsulfonyl, or (C₁-C₄)acylamino;
each of R⁶ and R¹⁰ is independently hydrogen, deuterium, halo, (C₁-C₃)alkyl, perfluoro(C₁-C₃)alkyl, hydroxy, (C₁-C₃)alkoxy, perfluoro(C₁-C₃)alkoxy, or amino;
each of R⁷ and R⁹ is independently hydrogen, deuterium, hydroxy, cyano, amino, halogen, halo(C₁-C₄)alkyl, (C₁-C₄)alkoxy, halo(C₁-C₄)alkoxy,

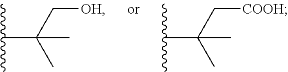

R⁸ is hydrogen, deuterium, halogen, halo(C₁-C₄)alkyl, (C₁-C₄)alkyl, (C₁-C₄)alkoxy, halo(C₁-C₄)alkoxy, cyano, phenyl, phenoxy, benzyloxy, amino,

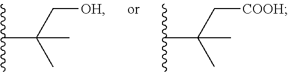

R³⁰ is (C₁-C₁₀)hydrocarbyl, (C₁-C₁₀)hydrocarbyl substituted with amino, (C₁-C₁₀)hydrocarbyl substituted with (C₁-C₄)hydrocarbyl, (C₁-C₁₀)hydrocarbyl substituted with carboxyl, carboxy, (C₁-C₆)alkoxycarbonyl, (C₁-C₆)alkoxycarbonylamino, methylthio, heterocyclyl, (C₁-C₁₀)oxaalkyl, CHR⁴⁴NHR⁴⁵ and guanidine;
each of R⁴⁰ and R⁴¹ is independently hydrogen or (C₁-C₆)hydrocarbyl;
R⁴² is (C₁-C₅)alkyl;
R⁴³ is (C₁-C₃)alkyl,
R⁴⁴ is a naturally occurring amino acid sidechain;
R⁴⁵ is H, methyl, or (C₁-C₄)alkoxycarbonyl; and
R⁵⁰ is H or (C₁-C₃)alkyl.

2. A method for increasing lysosomal acidity in a subject, comprising administering to the subject an amount of an agent which is a compound of Formula (I):

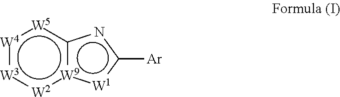

or a salt, hydrate, deuterated analog, or fluorinated analog thereof effective to increase lysosomal acidity in the subject, wherein:

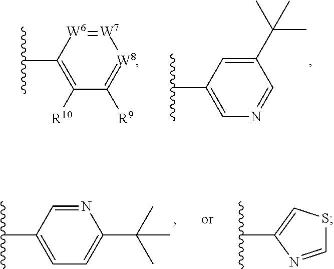

Ar is
W¹ is N—R¹, O, or S, or when W⁹ is N, W¹ may additionally be C—R⁵⁰;
W² is C—R² or N;
W³ is C—R³ or N;
W⁴ is C—R⁴ or N;
W⁵ is C—R⁵ or N;
W⁶ is C—R⁶ or N;
W⁷ is C—R⁷ or N;
W⁸ is C—R⁸ or N;
W⁹ is C, or when W¹ is C—R⁵⁰, W⁹ may be N;
R¹ is H, (C₁-C₃)alkyl, CH₂OC(═O)R³⁰, CH₂OP(═O)OR⁴⁰OR⁴¹, C(═O)OR⁴², or C(═O)R⁴³;
each of R², R³, R⁴, and R⁵ is independently hydrogen, deuterium, halogen, perfluoro(C₁-C₄)alkyl, (C₁-C₄)alkyl, (C₁-C₄)alkoxy, perfluoro(C₁-C₄)alkoxy, (C₁-C₄)acyl, (C₁₋₄)alkoxy(C₁-C₄)alkyl, hydroxy(C₁-C₄)alkyl, hydroxy, carboxy, (C₁-C₄)alkoxycarbonylamino, carboxamido, (C₁-C₄)alkylaminocarbonyl, cyano, acetoxy, nitro, amino, (C₁-C₄)alkylamino, di(C₁-C₄)alkylamino, mercapto, (C₁-C₄)alkylthio, aminosulfonyl, (C₁-C₄)alkylsulfonyl, or (C₁-C₄)acylamino;
each of R⁸ and R¹⁰ is independently hydrogen, deuterium, halo, (C₁-C₃)alkyl, perfluoro(C₁-C₃)alkyl, hydroxy, (C₁-C₃)alkoxy, perfluoro(C₁-C₃)alkoxy, or amino;
each of R⁷ and R⁹ is independently hydrogen, deuterium, hydroxy, cyano, amino, halogen, halo(C₁-C₄)alkyl, (C₁-C₄)alkoxy, halo(C₁-C₄)alkoxy,

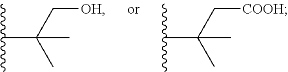

R⁸ is hydrogen, deuterium, halogen, halo(C₁-C₄)alkyl, (C₁-C₄)alkyl, (C₁-C₄)alkoxy, halo(C₁-C₄)alkoxy, cyano, phenyl. phenoxy, benzyloxy, amino,

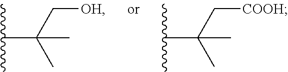

R³⁰ is (C₁-C₁₀)hydrocarbyl, (C₁-C₁₀)hydrocarbyl substituted with amino, (C₁-C₁₀)hydrocarbyl substituted with (C₁-C₄)hydrocarbyl, (C₁-C₁₀)hydrocarbyl substituted with carboxyl, carboxy, (C₁-C₆)alkoxycarbonyl, (C₁-C₆)alkoxycarbonylamino, methylthio, heterocycyl, (C₁-C₁₀)oxaalkyl, CHR⁴⁴NHR⁴⁵ and guanidine;
each of R⁴⁰ and R⁴¹ is independently hydrogen or (C₁-C₆)hydrocarbyl;
R⁴² is (C₁-C₅)alkyl;
R⁴³ is (C₁-C₃)alkyl,
R⁴⁴ is a naturally occurring amino acid sidechain;
R⁴⁵ is H, methyl, or (C₁-C₄)alkoxycarbonyl; and
R⁵⁰ is H or (C₁-C₃)alkyl.

3. A method of treating a subject having sepsis, an infection, or a disease or disorder associated with V-ATPase dysfunction, comprising administering to the subject a therapeutically effective amount of an agent which is a compound of Formula (I):

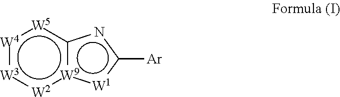

or a salt, hydrate, deuterated analog, or fluorinated analog thereof, wherein:

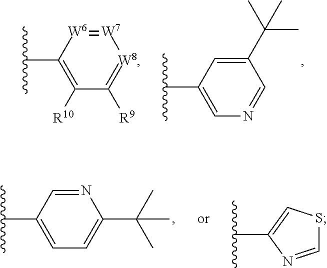

Ar is
W¹ is N—R¹, O, or S, or when W⁹ is N, W¹ may additionally be C—R⁵⁰;
W² is C—R² or N;
W³ is C—R³ or N;
W⁴ is C—R⁴ or N;
W⁵ is C—R⁵ or N;
W⁶ is C—R⁶ or N;
W⁷ is C—R⁷ or N;
W⁸ is C—R⁸ or N;
W⁹ is C, or when W¹ is C—R⁵⁰, W⁹ may be N;
R¹ is H, (C₁-C₃)alkyl, CH₂OC(═O)R³⁰, CH₂OP(═O)OR⁴⁰OR⁴¹, C(═O)OR⁴², or C(═O)R⁴³;
each of R², R³, R⁴, and R⁵ is independently hydrogen, deuterium, halogen, perfluoro(C₁-C₄)alkyl, (C₁-C₄)alkyl, (C₁-C₄)alkoxy, perfluoro(C₁-C₄)alkoxy, (C₁-C₄)acyl, (C₁₋₄)alkoxy(C₁-C₄)alkyl, hydroxy(C₁-C₄)alkyl, hydroxy, carboxy, (C₁-C₄)alkoxycarbonylamino, carboxamido, (C₁-C₄)alkylaminocarbonyl, cyano, acetoxy, nitro, amino, (C₁-C₄)alkylamino, di(C₁-C₄)alkylamino, mercapto, (C₁-C₄)alkylthio, aminosulfonyl, (C₁-C₄)alkylsulfonyl, or (C₁-C₄)acylamino;
each of R⁶ and R¹⁰ is independently hydrogen, deuterium, halo, (C₁-C₃)alkyl, perfluoro(C₁-C₃)alkyl, hydroxy, (C₁-C₃)alkoxy, perfluoro(C₁-C₃)alkoxy, or amino;
each of R⁷ and R⁹ is independently hydrogen, deuterium, hydroxy, cyano, amino, halogen, halo(C₁-C₄)alkyl, (C₁-C₄)alkoxy, halo(C₁-C₄)alkoxy,

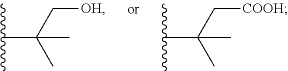

R⁸ is hydrogen, deuterium, halogen, halo(C₁-C₄)alkyl, (C₁-C₄)alkyl, (C₁-C₄)alkoxy, halo(C₁-C₄)alkoxy, cyano, phenyl, phenoxy, benzyloxy, amino,

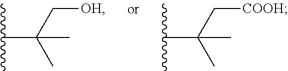

R³⁰ is (C₁-C₁₀)hydrocarbyl, (C₁-C₁₀)hydrocarbyl substituted with amino, (C₁-C₁₀)hydrocarbyl substituted with (C₁-C₄)hydrocarbyl, (C₁-C₁₀)hydrocarbyl substituted with carboxyl, carboxy, (C₁-C₆)alkoxycarbonyl, (C₁-C₆)alkoxycarbonylamino, methylthio, heterocycyl, (C₁-C₁₀)oxaalkyl, CHR⁴⁴NHR⁴⁵ and guanidine;
each of R⁴⁰ and R⁴¹ is independently hydrogen or (C₁-C₆)hydrocarbyl;
R⁴² is (C₁-C₅)alkyl;
R⁴³ is (C₁-C₃)alkyl,
R⁴⁴ is a naturally occurring amino acid sidechain;
R⁴⁵ is H, methyl, or (C₁-C₄)alkoxycarbonyl; and
R⁵⁰ is H or (C₁-C₃)alkyl.

4. The method of any one of embodiments 1 to 3, wherein the amount of the agent is an amount effective to increase lysosome acidity in the subject.

5. The method of any one of embodiments 1 to 4, wherein the amount of the agent is an amount effective to increase lysosome acidity in phagocytic cells of the subject (e.g., macrophages, monocytes, or neutrophils).

6. The method of any one of embodiments 1 to 5, wherein the amount of the agent is an amount effective to increase lysosome acidity in peritoneal cells of the subject.

7. The method of any one of embodiments 1 to 6, wherein the amount of the agent is an amount effective to increase lysosome acidity in peritoneal phagocytic cells of the subject (e.g., macrophages, monocytes, or neutrophils).

8. The method of any one of embodiments 1 to 7, wherein the amount of the agent is an amount effective to increase Tfeb mRNA levels in phagocytic cells of the subject (e.g., macrophages, monocytes, or neutrophils).

9. The method of any one of embodiments 1 to 8, wherein the amount of the agent is an amount effective to increase Tfeb mRNA levels in peritoneal cells of the subject, (e.g., phagocytic cells (e.g., macrophages, monocytes, or neutrophils)).

10. The method of any one of embodiments 1 to 9, wherein the amount of the agent is an amount effective to increase TFEB dephosphorylation in phagocytic cells of the subject (e.g., macrophages, monocytes, or neutrophils).

11. The method of any one of embodiments 1 to 10, wherein the amount of the agent is an amount effective to increase TFEB nuclear migration in phagocytic cells of the subject (e.g., macrophages, monocytes, or neutrophils).

12. The method of any one of embodiments 1 to 11, wherein the amount of the agent is an amount effective to increase the phosphorylated Akt to Akt ratio (p-Akt/Akt) in phagocytic cells of the subject (e.g., macrophages, monocytes, or neutrophils).

13. The method of any one of embodiments 1 to 12, wherein the amount of the agent is an amount effective to increase the phosphorylated PI3K to PI3K ratio (p-PI3K/PI3K) in phagocytic cells of the subject (e.g., macrophages, monocytes, or neutrophils).

14. The method of any one of embodiments 1 to 13, wherein the amount of the agent is an amount effective to increase physical contact between mitochondria and lysosomes in phagocytic cells of the subject (e.g., macrophages, monocytes, or neutrophils).

15. The method of any one of embodiments 1 to 14, wherein the amount of the agent is an amount effective to increase spare respiratory capacity in phagocytic cells of the subject (e.g., macrophages, monocytes, or neutrophils).

16. The method of any one of embodiments 1 to 15, wherein the amount of the agent is an amount effective to increase glycolytic capacity in phagocytic cells of the subject (e.g., macrophages, monocytes, or neutrophils).

17. The method of any one of embodiments 1 to 16, wherein the amount of the agent is an amount effective to increase glycolytic reserve in phagocytic cells of the subject (e.g., macrophages, monocytes, or neutrophils).

18. The method of any one of embodiments 1 to 17, wherein the amount of the agent is an amount effective to increase lysosomal proteolysis in phagocytic cells of the subject (e.g., macrophages, monocytes, or neutrophils).

19. The method of any one of embodiments 1 to 18, wherein the amount of the agent is an amount effective to increase mRNA levels of one or more hydrolases and/or one or more membrane proteins in phagocytic cells of the subject (e.g., macrophages, monocytes, or neutrophils).

20. The method of any one of embodiments 1 to 19, wherein the amount of the agent is an amount effective to increase mRNA levels of Ctsd in phagocytic cells of the subject (e.g., macrophages, monocytes, or neutrophils).

21. The method of any one of embodiments 1 to 20, wherein the amount of the agent is an amount effective to increase mRNA levels of Atp6v1A in phagocytic cells of the subject (e.g., macrophages, monocytes, or neutrophils).

22. The method of any one of embodiments 1 to 21, wherein the amount of the agent is an amount effective to increase mRNA levels of Atp6vOdi in phagocytic cells of the subject (e.g., macrophages, monocytes, or neutrophils).

23. The method of any one of embodiments 1 to 22, wherein the amount of the agent is an amount effective to increase mRNA levels of Mcoln1 in phagocytic cells of the subject (e.g., macrophages, monocytes, or neutrophils).

24. The method of any one of embodiments 1 to 22, wherein the amount of the agent is an amount effective to reduce the level of one or more markers of inflammation (e.g., in blood or serum).

25. The method of embodiment 24, wherein the one or more markers of inflammation comprise Tnfα, IL1β, IL6, IFNγ, or a combination thereof.

26. The method of any one of embodiments 1 to 25, wherein the subject does not have systemic immune activation.

27. The method of any one of embodiments 1 to 26, wherein the subject has suppressed innate immune function.

28. The method of embodiment 27, wherein the amount of the agent is an amount effective to enhance the subject's innate immune function.

29. The method of any one of embodiments 1 to 28, wherein the subject has suppressed macrophage phagocytic activity.

30. The method of embodiment 29, wherein the amount of the agent is an amount effective to enhance the subject's macrophage phagocytic activity.

31. The method of any one of embodiments 1 to 30, wherein the subject has a bowel perforation.

32. The method of any one of embodiments 1 to 31, wherein the subject has sepsis.

33. The method of embodiment 32, wherein the subject has polymicrobial sepsis.

34. The method of embodiment 32, wherein the subject has unimicrobial sepsis.

35. The method of any one of embodiments 32 to 34, wherein the subject has early sepsis.

36. The method of any one of embodiments 1 to 35, wherein the subject has a Sequential Organ Failure Assessment (SOFA) score of 0 to 6 prior to treatment with the agent, e.g., 0 to 3, 1 to 4, 2 to 5, 0, 1, 2, 3, 4, 5, or 6.

37. The method of any one of embodiments 1 to 35, wherein the subject has a Sequential Organ Failure Assessment (SOFA) score of 0 to 2 prior to treatment with the agent, e.g., 0, 1, or 2.

38. The method of any one of embodiments 1 to 35, wherein the subject has a Sequential Organ Failure Assessment (SOFA) score of 2 to 6 prior to treatment with the agent, e.g., 2, 3, 4, 5, or 6.

39. The method of any one of embodiments 1 to 35, wherein the subject has a Sequential Organ Failure Assessment (SOFA) score of 7 to 9 prior to treatment with the agent, e.g., 7, 8, or 9.

40. The method of any one of embodiments 1 to 35, wherein the subject has a Sequential Organ Failure Assessment (SOFA) score of 10 to 12 prior to treatment with the agent, e.g., 10, 11, or 12.

41. The method of any one of embodiments 1 to 35, wherein the subject has a Sequential Organ Failure Assessment (SOFA) score of 13 to 14 prior to treatment with the agent, e.g., 13 or 14.

42. The method of any one of embodiments 1 to 35, wherein the subject has a Sequential Organ Failure Assessment (SOFA) score of 15 to 24 prior to treatment with the agent, e.g., 15 to 20, 20 to 24, 15, 16, 17, 18, 19, 20, 21, 22, 23, or 24.

43. The method of any one of embodiments 1 to 42, wherein the amount of the agent is an amount effective to reduce the subject's SOFA score.

44. The method of any one of embodiments 1 to 42, wherein the amount of the agent is an amount effective to maintain the subject's SOFA score.

45. The method of any one of embodiments 1 to 44, wherein the subject has one, two, three, or four of (a)-(d):
(a) lower than normal levels of HLA-DR expression in the peripheral blood;
(b) higher than normal PD-1 expression in T cells;
(c) lower than normal CD88 expression in neutrophils;
(d) a lower than normal Thi7/Treg ratio.

46. The method of embodiment 45, wherein the subject has lower than normal levels of HLA-DR expression in the peripheral blood.

47. The method of embodiment 45 or 46, wherein the frequency of HLA-DR-expressing mononuclear cells in the peripheral blood of the subject is below 30%.

48. The method of any one of embodiments 45 to 47, wherein the subject has higher than normal PD-1 expression in T cells.

49. The method of any one of embodiments 45 to 48, wherein the subject has lower than normal CD88 expression in neutrophils.

50. The method of any one of embodiments 45 to 49, wherein the subject has a lower than normal Th17/Treg ratio.

51. The method of any one of embodiments 1 to 50, the subject has suppressed innate immune function and/or does not have systemic immune activation.

52. The method of any one of embodiments 1 to 51, wherein the subject has an infection.

53. The method of embodiment 52, wherein the subject has an infection of the abdominal cavity.

54. The method of embodiment 52 or embodiment 53, wherein the infection is a bacterial infection.

55. The method of embodiment 54, wherein the administration reduces the subject's bacterial load.

56. The method of embodiment 54 or embodiment 55, wherein the bacterial infection is a multi-drug resistant bacterial infection.

57. The method of embodiment 52 or embodiment 53, wherein the infection is a fungal infection.

58. The method of embodiment 57, wherein the administration reduces the subject's fungal load.

59. The method of embodiment 52 or embodiment 53, wherein the infection is a parasitic infection.

60. The method of embodiment 59, wherein the administration reduces the subject's parasitic load.

61. The method of embodiment 52 or embodiment 53, wherein the infection is a viral infection.

62. The method of embodiment 61, wherein the administration reduces the subject's viral load.

63. The method of any one of embodiments 1 to 62, wherein the subject has a V-ATPase dysfunction.

64. The method of any one of embodiments 1 to 63, wherein the subject has a disease or disorder associated with V-ATPase dysfunction.

65. The method of embodiment 64, wherein the disease or disorder associated with V-ATPase dysfunction is renal tubular acidosis, Zimmermann-Laband syndrome, Cutis laxa type II or wrinkly skin syndrome, osteopetrosis, glucose intolerance, diabetes, Parkinson's disease, Alzheimer's disease, or hearing loss.

66. The method of any one of embodiments 63 to 65, wherein the subject has a pathogenic mutation in a gene encoding a V-ATPase subunit.

67. The method of any one of embodiments 63 to 66, wherein the subject has a pathogenic mutation in a gene encoding a V-ATPase subunit B.

68. The method of embodiment 67, wherein the subject has a pathogenic ATP6V1B1 mutation.

69. The method of embodiment 63 or embodiment 68, wherein the subject has renal tubular acidosis.

70. The method of any one of embodiments 63 to 66, wherein the subject has a pathogenic ATP6V1B2 mutation.

71. The method of embodiment 63 or embodiment 70, wherein the subject has Zimmermann-Laband syndrome.

72. The method of any one of embodiments 63 to 66, wherein the subject has a pathogenic mutation in a gene encoding a V-ATPase subunit a.

73. The method of embodiment 72, wherein the subject has a pathogenic ATP6V0A4 mutation.

74. The method of embodiment 63 or embodiment 73, wherein the subject has renal tubular acidosis.

75. The method of embodiment 72, wherein the subject has a pathogenic ATP6V0A2 mutation.

76. The method of embodiment 63 or embodiment 75, wherein the subject has Cutis laxa type II or wrinkly skin syndrome.

77. The method of embodiment 72, wherein the subject has a pathogenic ATP6V0A3 mutation.

78. The method of embodiment 63 or embodiment 77, wherein the subject has osteopetrosis.

79. The method of embodiment 78, wherein the subject has neurodegeneration.

80. The method of any one of embodiments 63 to 66, wherein the subject has a pathogenic ATP6V1H mutation.

81. The method of embodiment 63 or embodiment 80, wherein the subject has glucose intolerance or diabetes.

82. The method of any one of embodiments 63 to 65, wherein the subject has a pathogenic mutation in a gene encoding a V-ATPase accessory protein.

83. The method of embodiment 82, wherein the V-ATPase accessory protein is ATP6AP2.

84. The method of embodiment 83, wherein the subject has Parkinson's disease, e.g., X-linked Parkinson Disease with Spacticity (XPDS).

85. The method of any one of embodiments 63 to 65, wherein the subject has a pathogenic PSEN1 mutation.

86. The method of embodiment 63 or embodiment 85, wherein the subject has familial Alzheimer's disease.

87. The method of any one of embodiments 63 to 65, wherein the subject has a pathogenic DMXL2 mutation.

88. The method of embodiment 63 or embodiment 87, wherein the subject has hearing loss.

89. The method of any one of embodiments 1 to 64, wherein the subject has renal tubular acidosis, Zimmermann-Laband syndrome, Cutis laxa type II or wrinkly skin syndrome, osteopetrosis, glucose intolerance, diabetes, Parkinson's disease, Alzheimer's disease, or hearing loss.

90. The method of any one of embodiments 1 to 89, wherein the agent is

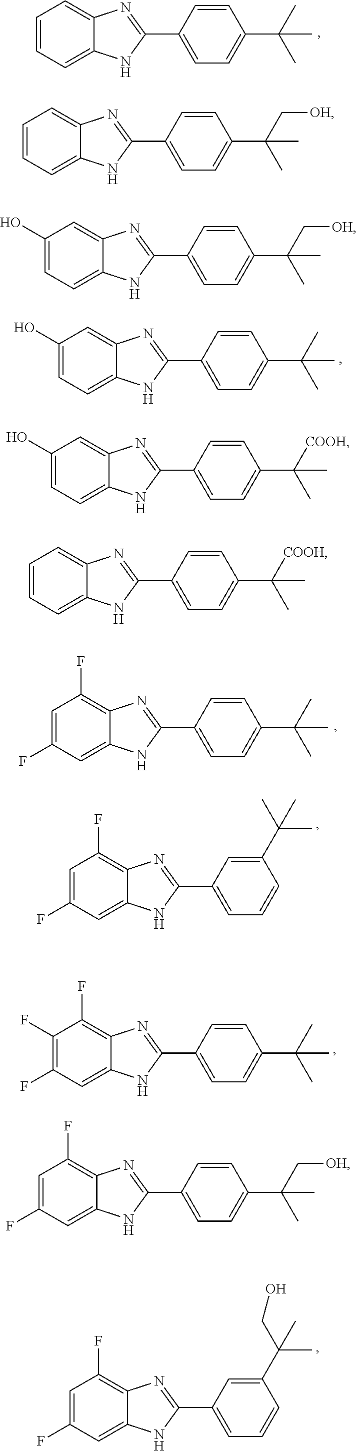

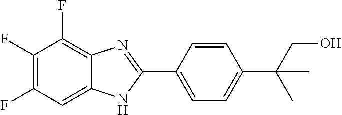 [SMILES: CC(C)(CO)c1ccc(-c2nc3c(F)c(F)c(F)cc3[nH]2)cc1]

or a salt, hydrate, deuterated analog, or fluorinated analog thereof.

91. The method of embodiment 90, wherein the agent is

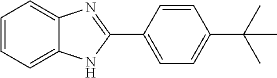 [SMILES: CC(C)(C)c1ccc(-c2nc3ccccc3[nH]2)cc1]

or a salt, hydrate, deuterated analog, or fluorinated analog thereof.

92. The method of embodiment 91, wherein the agent is

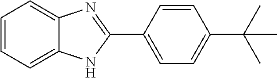 [SMILES: CC(C)(C)c1ccc(-c2nc3ccccc3[nH]2)cc1]

or a salt thereof.

93. The method of embodiment 90, wherein the agent is

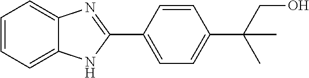 [SMILES: CC(C)(CO)c1ccc(-c2nc3ccccc3[nH]2)cc1]

or a salt, hydrate, deuterated analog, or fluorinated analog thereof.

94. The method of embodiment 90, wherein the agent is

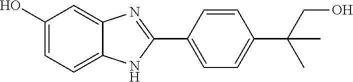 [SMILES: CC(C)(CO)c1ccc(-c2nc3cc(O)ccc3[nH]2)cc1]

or a salt, hydrate, deuterated analog, or fluorinated analog thereof.

95. The method of embodiment 90, wherein the agent is

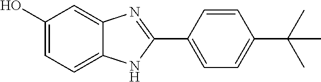 [SMILES: CC(C)(C)c1ccc(-c2nc3cc(O)ccc3[nH]2)cc1]

or a salt, hydrate, deuterated analog, or fluorinated analog thereof.

96. The method of embodiment 90, wherein the agent is

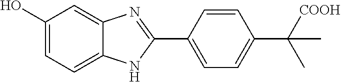 [SMILES: CC(C)(C(=O)O)c1ccc(-c2nc3cc(O)ccc3[nH]2)cc1]

or a salt, hydrate, deuterated analog, or fluorinated analog thereof. 97. The method of embodiment 90, wherein the agent is

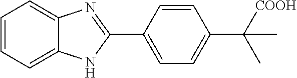 [SMILES: CC(C)(C(=O)O)c1ccc(-c2nc3ccccc3[nH]2)cc1]

or a salt, hydrate, deuterated analog, or fluorinated analog thereof.

98. The method of embodiment 90, wherein the agent is

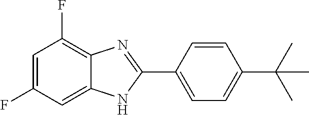 [SMILES: CC(C)(C)c1ccc(-c2nc3c(F)cc(F)cc3[nH]2)cc1]

or a salt, hydrate, deuterated analog, or fluorinated analog thereof.

99. The method of embodiment 90, wherein the agent is

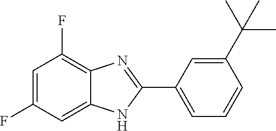 [SMILES: CC(C)(C)c1cccc(-c2nc3c(F)cc(F)cc3[nH]2)c1]

or a salt, hydrate, deuterated analog, or fluorinated analog thereof.

100. The method of embodiment 90, wherein the agent is

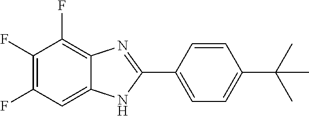 [SMILES: CC(C)(C)c1ccc(-c2nc3c(F)c(F)c(F)cc3[nH]2)cc1]

or a salt, hydrate, deuterated analog, or fluorinated analog thereof.

101. The method of embodiment 90, wherein the agent is

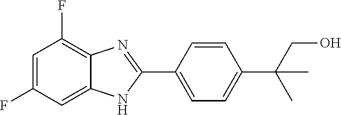 [SMILES: CC(C)(CO)c1ccc(-c2nc3c(F)cc(F)cc3[nH]2)cc1]

or a salt, hydrate, deuterated analog, or fluorinated analog thereof.

102. The method of embodiment 90, wherein the agent is

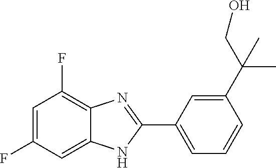 [SMILES: CC(C)(CO)c1cccc(-c2nc3c(F)cc(F)cc3[nH]2)c1]

or a salt, hydrate, deuterated analog, or fluorinated analog thereof.

103. The method of embodiment 90, wherein the agent is

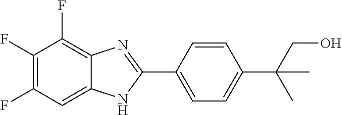 [SMILES: CC(C)(CO)c1ccc(-c2nc3c(F)c(F)c(F)cc3[nH]2)cc1]

or a salt, hydrate, deuterated analog, or fluorinated analog thereof.

104. The method of any one of embodiments 1 to 103, wherein the agent is administered enterically.

105. The method of embodiment 104, wherein the agent is administered by mouth.

106. The method of any one of embodiments 1 to 103, wherein the agent is administered by mouth (p.o.).

### 8. CITATION OF REFERENCES

All publications, patents, patent applications and other documents cited in this application are hereby incorporated by reference in their entireties for all purposes to the same extent as if each individual publication, patent, patent application or other document were individually indicated to be incorporated by reference for all purposes. In the event that there is an inconsistency between the teachings of one or more of the references incorporated herein and the present disclosure, the teachings of the present specification are intended.

What is claimed is:

1. A method of treating a subject having a disease or disorder associated with impaired lysosomal acidification, comprising administering to the subject a therapeutically effective amount of an agent which is a compound of Formula (I): 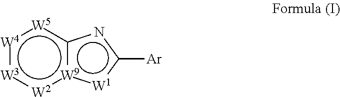
or a salt, hydrate, deuterated analog, or fluorinated analog thereof, wherein:
Ar is 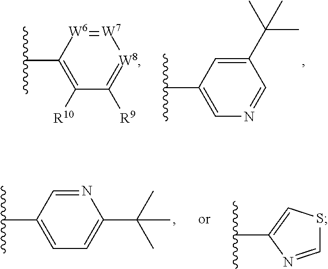
W¹ is N—R¹, O, or S, or when W⁹ is N, W¹ may additionally be C—R⁵⁰;
W² is C—R² or N;
W³ is C—R³ or N;
W⁴ is C—R⁴ or N;
W⁵ is C—R⁵ or N;
W⁶ is C—R⁶ or N;
W⁷ is C—R⁷ or N;
W⁸ is C—R⁸ or N;
W⁹ is C, or when W¹ is C—R⁵⁰, W⁹ may be N;
R¹ is H, (C₁-C₃)alkyl, CH₂OC(═O)R³⁰, CH₂OP(═O)OR⁴⁰OR⁴¹, C(═O)OR⁴², or C(═O)R⁴³;
each of R², R³, R⁴, and R⁵ is independently hydrogen, deuterium, halogen, perfluoro(C₁-C₄)alkyl, (C₁-C₄)alkyl, (C₁-C₄)alkoxy, perfluoro(C₁-C₄)alkoxy, (C₁-C₄)acyl, (C₁₋₄)alkoxy(C₁-C₄)alkyl, hydroxy(C₁-C₄)alkyl, hydroxy, carboxy, (C₁-C₄)alkoxycarbonylamino, carboxamido, (C₁-C₄)alkylaminocarbonyl, cyano, acetoxy, nitro, amino, (C₁-C₄)alkylamino, di(C₁-C₄)alkylamino, mercapto, (C₁-C₄)alkylthio, aminosulfonyl, (C₁-C₄)alkylsulfonyl, or (C₁-C₄)acylamino;
each of R⁶ and R¹⁰ is independently hydrogen, deuterium, halo, (C₁-C₃)alkyl, perfluoro(C₁-C₃)alkyl, hydroxy, (C₁-C₃)alkoxy, perfluoro(C₁-C₃)alkoxy, or amino;
each of R⁷ and R⁸ is independently hydrogen, deuterium, hydroxy, cyano, amino, halogen, halo(C₁-C₄)alkyl, (C₁-C₄)alkoxy, halo(C₁-C₄)alkoxy, 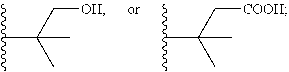
R⁸ is hydrogen, deuterium, halogen, halo(C₁-C₄)alkyl, (C₁-C₄)alkyl, (C₁-C₄)alkoxy, halo(C₁-C₄)alkoxy, cyano, phenyl, phenoxy, benzyloxy, amino, 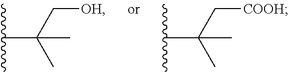
R³⁰ is (C₁-C₁₀)hydrocarbyl, (C₁-C₁₀)hydrocarbyl substituted with amino, (C₁-C₁₀)hydrocarbyl substituted with (C₁-C₄)hydrocarbyl, (C₁-C₁₀)hydrocarbyl substituted with carboxyl, carboxy, (C₁-C₆)alkoxycarbonyl, (C₁-C₆)alkoxycarbonylamino, methylthio, heterocyclyl, (C₁-C₁₀)oxaalkyl, CHR⁴⁴NHR⁴⁵ and guanidine;
each of R⁴⁰ and R⁴¹ is independently hydrogen or (C₁-C₆)hydrocarbyl;
R⁴² is (C₁-C₅)alkyl;
R⁴³ is (C₁-C₃)alkyl,
R⁴⁴ is a naturally occurring amino acid sidechain;
R⁴⁵ is H, methyl, or (C₁-C₄)alkoxycarbonyl; and
R⁵⁰ is H or (C₁-C₃)alkyl.

2. A method for increasing lysosomal acidity in a subject, comprising administering to the subject an amount of an agent which is a compound of Formula (I): 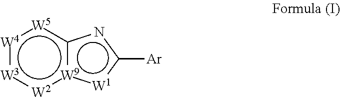
or a salt, hydrate, deuterated analog, or fluorinated analog thereof effective to increase lysosomal acidity in the subject, wherein:
Ar is 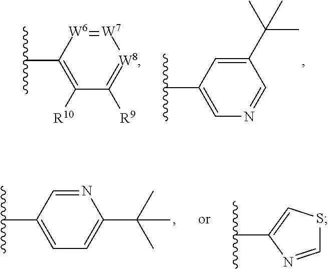
W¹ is N—R¹, O, or S, or when W⁹ is N, W¹ may additionally be C—R⁵⁰;
W² is C—R² or N;
W³ is C—R³ or N;
W⁴ is C—R⁴ or N;
W⁵ is C—R⁵ or N;
W⁶ is C—R⁶ or N;
W⁷ is C—R⁷ or N;
W⁸ is C—R⁸ or N;
W⁹ is C, or when W¹ is C—R⁵⁰, W⁹ may be N;
R¹ is H, (C₁-C₃)alkyl, CH₂OC(═O)R³⁰, CH₂OP(═O)OR⁴⁰OR⁴¹, C(═O)OR⁴², or C(═O)R⁴³;
each of R², R³, R4, and R⁵ is independently hydrogen, deuterium, halogen, perfluoro(C₁-C₄)alkyl, (C₁-C₄)alkyl, (C₁-C₄)alkoxy, perfluoro(C₁-C₄)alkoxy, (C₁-C₄)acyl, (C₁₋₄)alkoxy(C₁-C₄)alkyl, hydroxy(C₁-C₄)alkyl, hydroxy, carboxy, (C₁-C₄)alkoxycarbonylamino, carboxamido, (C₁-C₄)alkylaminocarbonyl, cyano, acetoxy, nitro, amino, (C₁-C₄)alkylamino, di(C₁-C₄)alkylamino, mercapto, (C₁-C₄)alkylthio, aminosulfonyl, (C₁-C₄)alkylsulfonyl, or (C₁-C₄)acylamino;
each of R⁶ and R¹⁰ is independently hydrogen, deuterium, halo, (C₁-C₃)alkyl, perfluoro(C₁-C₃)alkyl, hydroxy, (C₁-C₃)alkoxy, perfluoro(C₁-C₃)alkoxy, or amino;
each of R⁷ and R⁹ is independently hydrogen, deuterium, hydroxy, cyano, amino, halogen, halo(C₁-C₄)alkyl, (C₁-C₄)alkoxy, halo(C₁-C₄)alkoxy, 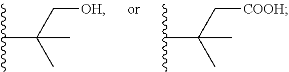
R⁸ is hydrogen, deuterium, halogen, halo(C₁-C₄)alkyl, (C₁-C₄)alkyl, (C₁-C₄)alkoxy, halo(C₁—C₄)alkoxy, cyano, phenyl, phenoxy, benzyloxy, amino, 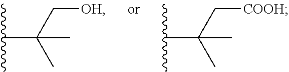
R³⁰ is (C₁-C₁₀)hydrocarbyl, (C₁-C₁₀)hydrocarbyl substituted with amino, (C₁-C₁₀)hydrocarbyl substituted with (C₁-C₄)hydrocarbyl, (C₁-C₁₀)hydrocarbyl substituted with carboxyl, carboxy, (C₁-C₆)alkoxycarbonyl, (C₁-C₆)alkoxycarbonylamino, methylthio, heterocycyl, (C₁-C₁₀)oxaalkyl, CHR⁴⁴NHR⁴⁵ and guanidine;
each of R⁴⁰ and R⁴¹ is independently hydrogen or (C₁-C₆)hydrocarbyl;
R⁴² is (C₁-C₅)alkyl;
R⁴³ is (C₁-C₃)alkyl,
R⁴⁴ is a naturally occurring amino acid sidechain;
R⁴⁵ is H, methyl, or (C₁-C₄)alkoxycarbonyl; and
R⁵⁰ is H or (C₁-C₃)alkyl.

3. A method of treating a subject having sepsis, an infection, or a disease or disorder associated with V-ATPase dysfunction, comprising administering to the subject a therapeutically effective amount of an agent which is a compound of Formula (I): 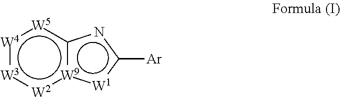
or a salt, hydrate, deuterated analog, or fluorinated analog thereof, wherein:
Ar is 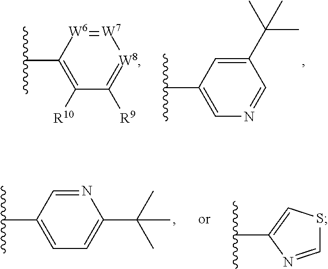
W¹ is N—R¹, O, or S, or when W⁹ is N, W¹ may additionally be C—R⁵⁰;
W² is C—R² or N;
W³ is C—R³ or N;
W⁴ is C—R⁴ or N;
W⁵ is C—R⁵ or N;
W⁶ is C—R⁶ or N;
W⁷ is C—R⁷ or N;
W⁸ is C—R⁸ or N;
W⁹ is C, or when W¹ is C—R⁵⁰, W⁹ may be N;
R¹ is H, (C₁-C₃)alkyl, CH₂OC(═O)R³⁰, CH₂OP(═O)OR⁴⁰OR⁴¹, C(═O)OR⁴², or C(═O)R⁴³;
each of R², R³, R⁴, and R⁵ is independently hydrogen, deuterium, halogen, perfluoro(C₁-C₄)alkyl, (C₁-C₄)alkyl, (C₁-C₄)alkoxy, perfluoro(C₁-C₄)alkoxy, (C₁-C₄)acyl, (C₁₋₄)alkoxy(C₁-C₄)alkyl, hydroxy(C₁-C₄)alkyl, hydroxy, carboxy, (C₁-C₄)alkoxycarbonylamino, carboxamido, (C₁-C₄)alkylaminocarbonyl, cyano, acetoxy, nitro, amino, (C₁-C₄)alkylamino, di(C₁-C₄)alkylamino, mercapto, (C₁-C₄)alkylthio, aminosulfonyl, (C₁-C₄)alkylsulfonyl, or (C₁-C₄)acylamino;
each of R⁶ and R¹⁰ is independently hydrogen, deuterium, halo, (C₁-C₃)alkyl, perfluoro(C₁-C₃)alkyl, hydroxy, (C₁-C₃)alkoxy, perfluoro(C₁-C₃)alkoxy, or amino;
each of R⁷ and R⁹ is independently hydrogen, deuterium, hydroxy, cyano, amino, halogen, halo(C₁-C₄)alkyl, (C₁-C₄)alkoxy, halo(C₁-C₄)alkoxy, 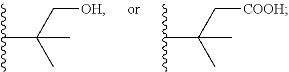
R⁸ is hydrogen, deuterium, halogen, halo(C₁-C₄)alkyl, (C₁-C₄)alkyl, (C₁-C₄)alkoxy, halo(C₁-C₄)alkoxy, cyano, phenyl, phenoxy, benzyloxy, amino, 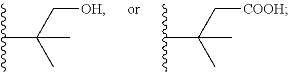
R³⁰ is (C₁-C₁₀)hydrocarbyl, (C₁-C₁₀)hydrocarbyl substituted with amino, (C₁-C₁₀)hydrocarbyl substituted with (C₁-C₄)hydrocarbyl, (C₁-C₁₀)hydrocarbyl substituted with carboxyl, carboxy, (C₁-C₆)alkoxycarbonyl, (C₁-C₆)alkoxycarbonylamino, methylthio, heterocyclyl, (C₁-C₁₀)oxaalkyl, CHR⁴⁴NHR⁴⁵ and guanidine;
each of R⁴⁰ and R⁴¹ is independently hydrogen or (C₁-C₆)hydrocarbyl;
R⁴² is (C₁-C₅)alkyl;
R⁴³ is (C₁-C₃)alkyl,
R⁴⁴ is a naturally occurring amino acid sidechain;
R⁴⁵ is H, methyl, or (C₁-C₄)alkoxycarbonyl; and
R⁵⁰ is H or (C₁-C₃)alkyl.

4. The method of any one of claims 1 to 3, wherein the amount of the agent is an amount effective to increase lysosome acidity in the subject.

5. The method of any one of claims 1 to 4, wherein the amount of the agent is an amount effective to increase lysosome acidity in phagocytic cells of the subject (e.g., macrophages, monocytes, or neutrophils).

6. The method of any one of claims 1 to 5, wherein the amount of the agent is an amount effective to increase lysosome acidity in peritoneal cells of the subject.

7. The method of any one of claims 1 to 6, wherein the amount of the agent is an amount effective to increase lysosome acidity in peritoneal phagocytic cells of the subject (e.g., macrophages, monocytes, or neutrophils).

8. The method of any one of claims 1 to 7, wherein the amount of the agent is an amount effective to:
(a) increase Tfeb mRNA levels in phagocytic cells of the subject (e.g., macrophages, monocytes, or neutrophils);
(b) increase Tfeb mRNA levels in peritoneal cells of the subject, (e.g., phagocytic cells (e.g., macrophages, monocytes, or neutrophils));
(c) increase TFEB dephosphorylation in phagocytic cells of the subject (e.g., macrophages, monocytes, or neutrophils);
(d) increase TFEB nuclear migration in phagocytic cells of the subject (e.g., macrophages, monocytes, or neutrophils);
(e) increase the phosphorylated Akt to Aid ratio (p-Akt/Akt) in phagocytic cells of the subject (e.g., macrophages, monocytes, or neutrophils);
(f) increase the phosphorylated PI3K to PI3K ratio (p-PI3K/PI3K) in phagocytic cells of the subject (e.g., macrophages, monocytes, or neutrophils);
(g) increase physical contact between mitochondria and lysosomes in phagocytic cells of the subject (e.g., macrophages, monocytes, or neutrophils);
(h) increase spare respiratory capacity in phagocytic cells of the subject (e.g., macrophages, monocytes, or neutrophils);
(i) increase glycolytic capacity in phagocytic cells of the subject (e.g., macrophages, monocytes, or neutrophils);
(j) increase glycolytic reserve in phagocytic cells of the subject (e.g., macrophages, monocytes, or neutrophils);
(k) increase lysosomal proteolysis in phagocytic cells of the subject (e.g., macrophages, monocytes, or neutrophils);
(l) increase mRNA levels of one or more hydrolases and/or one or more membrane proteins in phagocytic cells of the subject (e.g., macrophages, monocytes, or neutrophils);
(m) increase mRNA levels of Ctsd in phagocytic cells of the subject (e.g., macrophages, monocytes, or neutrophils);
(n) increase mRNA levels of Atp6v1A in phagocytic cells of the subject (e.g., macrophages, monocytes, or neutrophils);
(o) increase mRNA levels of Atp6v0d1 in phagocytic cells of the subject (e.g., macrophages, monocytes, or neutrophils);
(p) increase mRNA levels of Mcoln1 in phagocytic cells of the subject (e.g., macrophages, monocytes, or neutrophils); or
(q) reduce the level of one or more markers of inflammation (e.g., in blood or serum).

9. The method of any one of claims 1 to 8, wherein the subject does not have systemic immune activation.

10. The method of any one of claims 1 to 9, wherein the subject has suppressed innate immune function, optionally wherein the amount of the agent is an amount effective to enhance the subject's innate immune function.

11. The method of any one of claims 1 to 10, wherein the subject has suppressed macrophage phagocytic activity, optionally wherein the amount of the agent is an amount effective to enhance the subject's macrophage phagocytic activity.

12. The method of any one of claims 1 to 11, wherein the subject has a bowel perforation.

13. The method of any one of claims 1 to 12, wherein the subject has sepsis, e.g., polymicrobial sepsis or unimicrobial sepsis.

14. The method of claim 13, wherein the subject has early sepsis.

15. The method of any one of claims 1 to 14, wherein the subject has an infection.

16. The method of claim 15, wherein the infection is a bacterial infection, a fungal infection, a parasitic infection, or a viral infection.

17. The method of any one of claims 1 to 16, wherein the subject has a V-ATPase dysfunction.

18. The method of any one of claims 1 to 17, wherein the subject has a disease or disorder associated with V-ATPase dysfunction.

19. The method of claim 18, wherein the disease or disorder associated with V-ATPase dysfunction is renal tubular acidosis, Zimmermann-Laband syndrome, Cutis laxa type II or wrinkly skin syndrome, osteopetrosis, glucose intolerance, diabetes, Parkinson's disease, Alzheimer's disease, or hearing loss.

20. The method of any one of claims 17 to 19, wherein the subject has a pathogenic mutation in a gene encoding a V-ATPase subunit.

21. The method of any one of claims 17 to 20, wherein the subject has a pathogenic mutation in a gene encoding a V-ATPase subunit B, optionally wherein the subject has a pathogenic ATP6V1B1 mutation.

22. The method of claim 21, wherein the subject has a pathogenic ATP6V1B1 mutation.

23. The method of claim 17 or claim 22, wherein the subject has renal tubular acidosis.

24. The method of any one of claims 17 to 20, wherein the subject has a pathogenic ATP6V1B2 mutation.

25. The method of claim 17 or claim 24, wherein the subject has Zimmermann-Laband syndrome.

26. The method of any one of claims 17 to 20, wherein the subject has a pathogenic mutation in a gene encoding a V-ATPase subunit a.

27. The method of claim 26, wherein the subject has a pathogenic ATP6V0A4 mutation.

28. The method of claim 17 or claim 27, wherein the subject has renal tubular acidosis.

29. The method of claim 26, wherein the subject has a pathogenic ATP6V0A2 mutation.

30. The method of claim 17 or claim 29, wherein the subject has Cutis laxa type II or wrinkly skin syndrome.

31. The method of claim 26, wherein the subject has a pathogenic ATP6V0A3 mutation.

32. The method of claim 17 or claim 31, wherein the subject has osteopetrosis.

33. The method of claim 32, wherein the subject has neurodegeneration.

34. The method of any one of claims 17 to 20, wherein the subject has a pathogenic ATP6V1H mutation.

35. The method of claim 17 or claim 34, wherein the subject has glucose intolerance or diabetes.

36. The method of any one of claims 17 to 19, wherein the subject has a pathogenic mutation in a gene encoding a V-ATPase accessory protein.

37. The method of claim 36, wherein the V-ATPase accessory protein is ATP6AP2.

38. The method of claim 37, wherein the subject has Parkinson's disease, e.g., X-linked Parkinson Disease with Spacticity (XPDS).

39. The method of any one of claims 17 to 19, wherein the subject has a pathogenic PSEN1 mutation.

40. The method of claim 17 or claim 39, wherein the subject has familial Alzheimer's disease.

41. The method of any one of claims 17 to 19, wherein the subject has a pathogenic DMXL2 mutation.

42. The method of claim 17 or claim 41, wherein the subject has hearing loss.

43. The method of any one of claims 1 to 18, wherein the subject has renal tubular acidosis, Zimmermann-Laband syndrome, Cutis laxa type II or wrinkly skin syndrome, osteopetrosis, glucose intolerance, diabetes, Parkinson's disease, Alzheimer's disease, or hearing loss.

44. The method of any one of claims 1 to 43, wherein the agent is 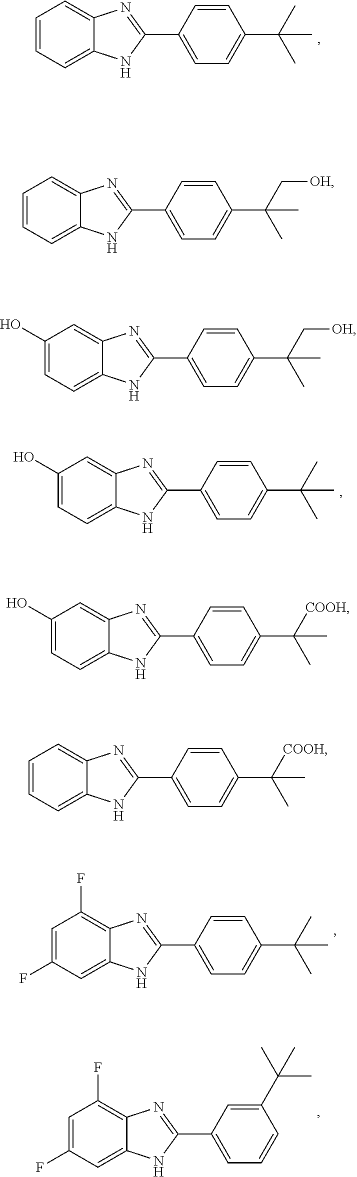 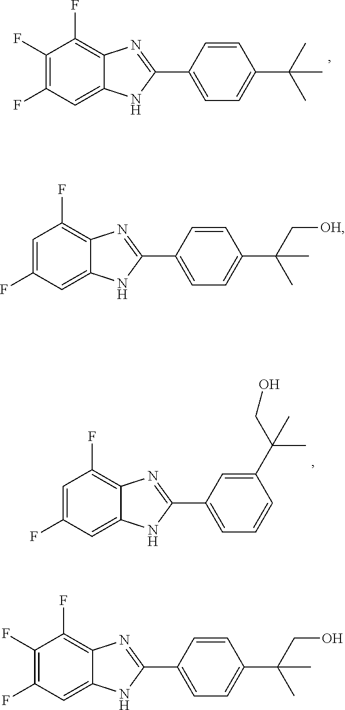 [SMILES: CC(C)(C)c1ccc(-c2nc3c(F)c(F)c(F)cc3[nH]2)cc1 ; CC(C)(CO)c1ccc(-c2nc3c(F)cc(F)cc3[nH]2)cc1 ; CC(C)(CO)c1cccc(-c2nc3c(F)cc(F)cc3[nH]2)c1 ; CC(C)(CO)c1ccc(-c2nc3c(F)c(F)c(F)cc3[nH]2)cc1]
or a salt, hydrate, deuterated analog, or fluorinated analog thereof.

45. The method of claim 44, wherein the agent is 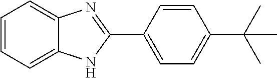 [SMILES: CC(C)(C)c1ccc(-c2nc3ccccc3[nH]2)cc1]
or a salt, hydrate, deuterated analog, or fluorinated analog thereof.

46. The method of claim 45, wherein the agent is 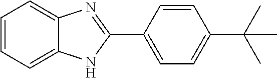 [SMILES: CC(C)(C)c1ccc(-c2nc3ccccc3[nH]2)cc1]
or a salt thereof.

47. The method of any one of claims 1 to 46, wherein the agent is administered enterically.

48. The method of claim 47, wherein the agent is administered by mouth.

49. The method of any one of claims 1 to 46, wherein the agent is administered by mouth (p.o.).
